(19) United States
(12) Patent Application Publication
(10) Pub. No.: US 2018/0108430 A1
(43) Pub. Date: Apr. 19, 2018

(54) METHOD AND SYSTEM FOR POPULATION HEALTH MANAGEMENT IN A CAPTIVATED HEALTHCARE SYSTEM

(71) Applicants: Alexander H. VO, Denver, CO (US); Stephen Smock, Katy, TX (US); Anthony K. Williams, Willis, TX (US); Ben Raimer, Galveston, TX (US); Owen J. Murray, Houston, TX (US); Ryan S. Westberry, Denver, CO (US); David L. Callender, Galveston, TX (US); Kelly Michael Jackson, San Antonio, TX (US)

(72) Inventors: Alexander H. VO, Denver, CO (US); Stephen Smock, Katy, TX (US); Anthony K. Williams, Willis, TX (US); Ben Raimer, Galveston, TX (US); Owen J. Murray, Houston, TX (US); Ryan S. Westberry, Denver, CO (US); David L. Callender, Galveston, TX (US); Kelly Michael Jackson, San Antonio, TX (US)

(73) Assignee: Board of Regents, The University of Texas System, Austin, TX (US)

(21) Appl. No.: 15/719,382

(22) Filed: Sep. 28, 2017

Related U.S. Application Data
(60) Provisional application No. 62/402,493, filed on Sep. 30, 2016.

(51) Int. Cl. G16H 10/60; G06F 17/30; G09B 5/06; G09B 19/00; G16H 50/30; G16H 40/67

(52) U.S. Cl. CPC G16H 10/60; G06F 17/30864; G16H 40/67; G09B 19/00; G16H 50/30; G09B 5/065

(57) ABSTRACT

Methods and systems are provided for managing patient or patient populations within a fully integrated captivated healthcare system, which includes creating a patient profile or a patient population profile; screening, assessing and treating a patient or patient population; reviewing, analyzing, and comparing a patient or patient population to a control group or pre-treatment patient profile or patient population profile; and prioritizing, scheduling, and care coordination for a patient or patient population as part of follow-up healthcare assessment and services included in the fully integrated healthcare delivery system. Part of the methods and system includes the ability to utilize comparative patient and patient population data to assess and control healthcare access, quality, and costs.

### CROSS-REFERENCE TO RELATED APPLICATIONS

This patent application claims priority to U.S. provisional patent application Ser. No. 62/402,493 filed on Sep. 30, 2016 entitled “Method and System for Population Health Management in a Captivated Healthcare System”, which is hereby incorporated by reference in its entirety.

### INCORPORATION-BY-REFERENCE OF MATERIALS FILED ON COMPACT DISC

None.

### TECHNICAL FIELD OF THE INVENTION

The present invention relates generally to the field of information systems and, more particularly, to managing patient or patient populations within a fully integrated captivated healthcare system.

### STATEMENT OF FEDERALLY FUNDED RESEARCH

None.

### BACKGROUND OF THE INVENTION

The medical needs of inmates in US prisons and jails were largely ignored by society until the early 1970s, when the convergence of several independent forces brought the issue of correctional healthcare to the forefront. One of these forces was organized medicine. In response to surveys indicating serious and pervasive healthcare deficiencies within correctional institutions, several organizations became involved in efforts to improve medical care for prisoners. The American Public Health Association published the first comprehensive national healthcare standards for correctional facilities in 1976. The American Medical Association developed model healthcare delivery systems for jails, published its own correctional healthcare standards, and established a program to accredit healthcare systems in jails.

Another force in bringing about reforms was the court system. A series of decisions by the lower federal courts during the early 1970s established a constitutional basis for providing an adequate level of medical care to prisoners. In the landmark case of Estelle v Gamble, the US Supreme Court affirmed that deliberate indifference to the serious medical needs of prisoners was a violation of the Eighth Amendment and ruled that the federal courts could intervene to ensure sufficient medical care. By 1981, 25 states were under court order or consent decree to improve conditions in their correctional institutions.

These reforms were soon threatened by demographic changes in the nation's correctional populations. Responding to society's concerns about crime, lawmakers enacted legislation that limited judicial discretion in sentencing for certain offenses, resulting in sizeable increases in the number of mandatory and fixed sentences, and concurrent decreases in the use of alternatives to incarceration. Between 1980 and the early 1990s, the US prison population more than doubled. Additionally, incarceration rates for offenders with special healthcare needs, including elderly offenders and women, increased rapidly.

The explosive growth of the prison population, coupled with soaring medical costs and a shrinking base of public funding, severely strained the healthcare resources of many correctional systems in the 1990s. Moreover, health policy analysts began to point out that existing health delivery models were not designed to handle the increase in chronic illnesses and infectious diseases within the prison population. Faced with these challenges and a legal mandate to improve conditions in its prisons, the state of Texas began to develop and implement a novel fully integrated population management healthcare system.

The Texas correctional managed healthcare system is structured on a series of contractual relationships between Texas Department of Criminal Justice (TDCJ), Correctional Managed Health Care Committee (CMHCC), and 2 state medical schools. TDCJ contracts with CMHCC to provide statewide oversight and coordination of health services. The CMHCC in turn contracts with University of Texas Medical Branch (UTMB) and Texas Tech University Health Sciences Center (TTUHSC) to provide physical, mental, and dental healthcare to the prison population. Both universities subcontract with local clinicians on an as-needed basis.

The TDCJ Health Services Division monitors the quality of care delivered by the contracted clinicians via its Health Services Quality Improvement Program. Biennial operational reviews of prison health facilities are conducted to ensure compliance with national and state standards and laws. Additionally, the division investigates all medical-related grievances, reviews all prisoner deaths, and monitors the incidence of communicable diseases.

The CMHCC is composed of 9 appointed members, including 3 public members and 2 representatives from TDCJ, UTMB, and TTUHSC. Five members are physicians. Besides coordinating the contractual provision of health services, the committee monitors the general quality of healthcare, resolves disputes related to medical care, and implements the use of case management, utilization review, and other managed care tools. The committee has the power to enforce compliance with contract provisions.

The TTUHSC service area includes the western portion of Texas (about 20% of the prison population); UTMB serves the remainder of the population. UTMB primarily uses its own employees to provide correctional health services; TTUHSC uses a mix of its own employees and subcontracted local clinicians.

Primary care is provided at ambulatory clinics located in every TDCJ facility. Basic dental and mental health services are also available at all but a few clinics. Additionally, infirmaries are located in 16 correctional facilities. More advanced levels of care are available at Hospital Galveston and several regional medical facilities. Either the American Correctional Association or the National Commission on Correctional Health Care accredits all medical facilities. Hospital Galveston has been awarded accreditation with full standards compliance by the Joint Commission on Accreditation of Healthcare Organizations.

A number of programs are available for inmates with special health needs, including those with chronic diseases and psychiatric disorders. Chronic care clinics have been established to provide more effective interventions for the increasing number of offenders with chronic diseases. Elements of chronic care management include patient education, regular evaluations by a clinician, monitoring for medication efficacy and compliance, and evaluation of laboratory tests. Interventions are based on evidence-based practice guidelines and clinical protocols developed by a joint pharmacy and therapeutics committee. The clinics provide care for patients with hepatitis C virus infection, essential hypertension, psychiatric disorders, asthma, diabetes mellitus, HIV infection, and numerous other diseases and disorders.

Many inmates have a medical alert code, indicating a current or past history of a mental disorder. Services available to this population include group and individual psychotherapy, psychopharmacologic therapy, and crisis intervention counseling. Inpatient care is provided at dedicated psychiatric facilities. Outpatient services are available at almost all ambulatory clinics. Extensive use of telepsychiatry has also expanded treatment access and scope. As an alternative to administrative segregation, TTUHSC implemented an inpatient program for mentally ill offenders with a history of aggression. The Program for the Aggressive Mentally-Ill Offender uses cognitive behavioral therapy to help inmates curb aggressive impulses. Texas also was one of the first states to develop a model program for the mentally retarded offender. Telehealth has been an integral component of healthcare delivery under the managed care model. Besides general medicine and surgery, 28 specialty and subspecialty services are available via Telehealth. Telehealth has proved to be especially effective for Texas' sprawling prison system, because it improves timely access to specialty care for offenders in remote locations. Telehealth also has helped reduce the overall cost of transporting inmates over considerable distances for consultations.

UTMB deploys a correctional telehealth network that combines the videoconferencing capabilities of telehealth with a customized electronic records management system (EMR). The technology enables secure, comprehensive storage of medical records that can be readily accessed by clinicians at the prisons and the hub site. Clinicians also have immediate access to the TDCJ formulary and disease management guidelines. Utilization review and case management are facilitated by the fully digital system, which also integrates a pharmacy management program to permit monitoring of compliance with treatment guidelines.

### SUMMARY OF THE INVENTION

The disclosed invention relates to a comprehensive integrated healthcare population management method and system in a captivated healthcare system, which includes creating a patient profile or a patient population profile; screening, assessing and treating a patient or patient population; reviewing, analyzing, and comparing a patient or patient population to a control group or pre-treatment patient profile or patient population profile; and prioritizing, scheduling, and care coordination for a patient or patient population as part of follow-up healthcare assessment and services included in the fully integrated healthcare delivery system. The disclosed invention further relates to the ability to utilize comparative patient and patient population data to assess and control healthcare access, quality and cost using aggregated and de-aggregated data displayed on a plurality of dashboards.

In one embodiment, a computerized method of population health management in a captivated healthcare system (a) provides a computer system having at least one memory unit and at least one processing unit communicably coupled to the at least one memory unit, wherein the at least one memory unit stores data in one or more patient profiles or patient population profiles, (b) screens one or more of the patients or patient population for a disease based on one or more established clinical protocols using the computer system, (c) assesses a state of the disease based upon clinical results, and updating the patient profile(s) or patient population profile(s) to include the clinical results of the at least one test and the assessed disease state using the computer system, (d) determines a health management status of the one or more patients or patient population in comparison to the established clinical protocol(s) or a disease criteria, a control patient or the patient population or other comparison mechanism using the computer system, and (e) provides a recommended corrective action plan (CAP) based on the health management status and patient profile(s) or patient population profile(s) using the computer system.

In other aspects or embodiments, the method may include the step of coordinating a delivery of a health care to a plurality of patients using the computer system, wherein the health care comprises an acute and non-acute integrated health care involving at least one of a physical health care, a behavioral health care, or a dental health care. Other steps may include (a) evaluating the delivery of the health care to the plurality of patients using the computer system based on one or more metrics comprising at least one of a patient access to the health care, a quality of the health care, a cost of the health care, a clinical data, a non-clinical data or a financial data, (b) coordinating a follow-up to the delivery of the health care to the plurality of patients using the computer system, or (c) providing one or more health education materials to the plurality of patients using the computer system. The patient profile(s) or patient population profile(s) may include at least one unique identifier such as medical record number, social security number, inmate number, unit number, facility name, region name, or state name. The patient profile(s) or patient population profile(s) may further includes a prior medical record(s) or a history of medical conditions.

In other aspects or embodiments, the screening step may further (a) include performing at least one screening test and including results of the at least one screening test in the patient profile(s) or patient population profile(s), (b) be based on the established clinical protocol and/or disease criteria, or (c) include an evaluation of risk factors in the patient profile(s) or patient population profile(s) relevant to the disease state or condition. The disease may be a first disease, and the steps further include determining a health management status of the patient with respect to a second disease based on the patient profile and a co-morbidity of the first disease with the second disease.

In other aspects or embodiments, the method may include (a) receiving an inquiry or request from a patient for related products, treatment, procedures, tests, education or counseling via the computer system, (b) providing a recommended action and a clinical or non-clinical follow-up via telehealth, (c) providing a recommended action to a recipient on an intranet or internet-based system, (d) displaying a recommended action on a visual enabled device comprising at least one of a computer, a smart phone, or tablet, (e) implementing the recommended corrective action plan (CAP) to address the disease state or condition, (f) providing the one or more metrics and one or more recommendations via one or more devices communicably coupled to the computer system, (g) displaying at least one of the metrics in a dashboard format on the one or more devices, or (h) providing a quality system and risk management program.

In another embodiment, a system of population health management in a captivated healthcare system includes at least one memory unit to store a patient profile or a patient population profile including a patient data and a test results, and at least one processing unit communicably coupled with at least one memory unit. The at least one processing unit (a) screens one or more of the patients or patient population for a disease based on one or more established clinical protocols, (b) assesses a state of the disease based upon clinical results, and updating the patient profile(s) or patient population profile(s) to include the clinical results of the at least one test and the assessed disease state, (c) determines a health management status of the one or more patients or patient population in comparison to the established clinical protocol(s) or a disease criteria, a control patient or the patient population or other comparison mechanism, and (d) provides a recommended corrective action plan (CAP) based on the health management status and patient profile(s) or patient population profile(s). In other aspects or embodiments, the at least one processing unit: can be communicably coupled to one or more internal databases or local devices; can be communicably coupled to one or more remote devices and/or external database(s) via a network; and/or can automatically receive data, transmit data or control medical-related devices. The system may perform other functionality and/or include other components as described above in reference to the method.

The present invention is described in detail below with reference to the accompanying drawings.

### BRIEF DESCRIPTION OF THE DRAWINGS

The above and further advantages of the invention may be better understood by referring to the following description in conjunction with the accompanying drawings, in which:

FIG. 1 is a hub and spoke diagram outlining the components of the method and system according to an embodiment of the current invention;

FIG. 2 is a workflow diagram outlining clinical decision points and analyses metrics according to an embodiment of the current invention;

FIG. 3 is a data flow diagram outlining the data transport, data storage, and data reporting according to an embodiment of the current invention;

FIG. 4 is a flow diagram outlining the population health management metrics calculation process.

FIG. 5 is a flow diagram outlining the multidimensional persistent data store KPI fact.

FIG. 6 is a flow diagram outlining the multidimensional persistent data store patient fact.

FIGS. 7-8 are examples of dashboard interfaces according to an embodiment of the current invention;

FIG. 9 is a flow diagram of the Quality Systems and Risk Management processes according to an embodiment of the current invention;

FIGS. 10-13 are annotated screen shots of the Medical Sick Call interfaces and printed output reminder according to an embodiment of the current invention;

FIG. 14 is an annotated screen shot of a patient level dashboard according to an embodiment of the current invention;

FIG. 15 is a screen shot of a Corrective Action Plan (CAP) form according to an embodiment of the current invention;

FIG. 16 is a screen shot of a facility level dashboard according to an embodiment of the current invention;

FIG. 17 is a screen shot of a region level dashboard according to an embodiment of the current invention;

FIG. 18 is a screen shot of an all region level dashboard according to an embodiment of the current invention;

FIG. 19 is a list of patent education topics according to an embodiment of the current invention;

FIGS. 20-21 are examples of dashboard interfaces according to another embodiment of the current invention;

FIG. 22 is a screen shot of a facility level dashboard according to another embodiment of the current invention;

FIG. 23 is a screen shot of a region level dashboard according to another embodiment of the current invention;

FIG. 24 is a screen shot of an all region level dashboard according to another embodiment of the current invention;

FIG. 25 is a flow chart of a method in accordance with another embodiment of the current invention;

FIG. 26 is a block diagram of a system in accordance with another embodiment of the current invention; and

FIGS. 27A-27I are a diabetes mellitus clinical protocol according to an embodiment of the current invention.

### DETAILED DESCRIPTION OF THE INVENTION

The current invention now will be described more fully hereinafter with reference to the accompanying drawings, which illustrate embodiments of the invention. This invention may, however, be embodied in many different forms and should not be construed as limited to the illustrated embodiments set forth herein. For example, the present invention is not limited to use in a captivated healthcare system. Rather, these embodiments are provided so that this disclosure will be thorough and complete, and will fully convey the scope of the invention to those skilled in the art.

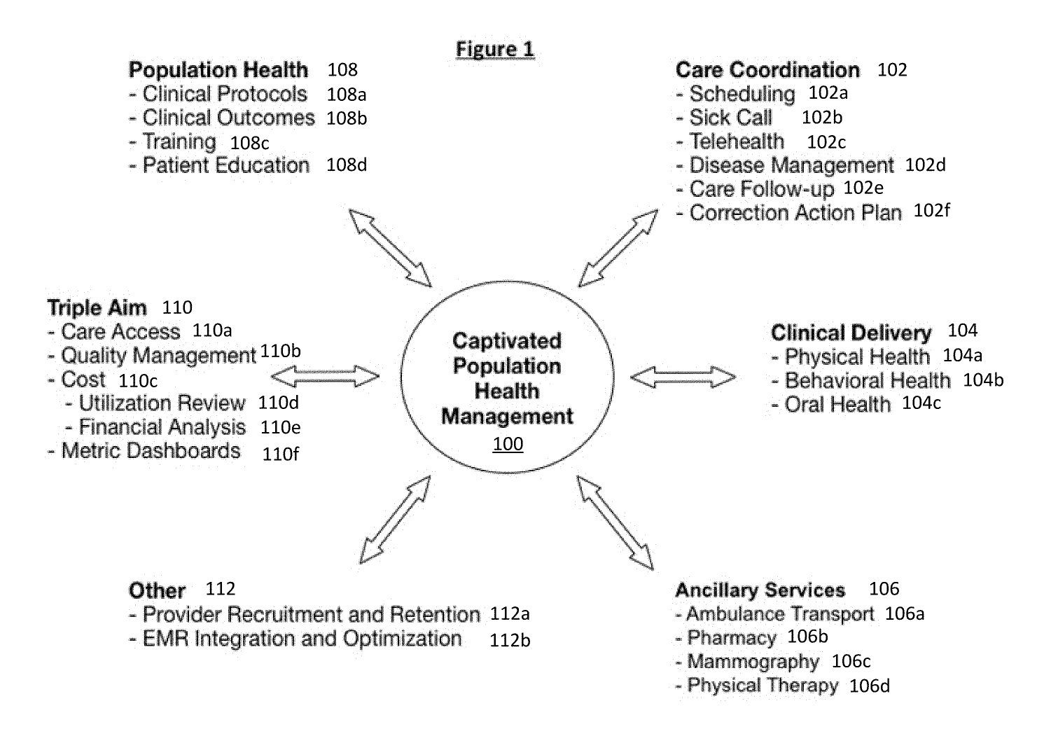

Embodiments of the current invention provide a method and system that provide population health management in a captivated healthcare system. FIG. 1 shows embodiments of the current invention that provide a plurality of services and support that directly and indirectly increase patient access to primary and specialty healthcare; healthcare quality metrics, evaluation and comparison, and reduction in overall healthcare costs. Further embodiments of the current invention are provided that improve patient outcomes, increase communication between key stakeholders, identify opportunities for process improvement and monitor clinical progress of patients.

Now referring to FIG. 1, a hub and spoke diagram summarizing the services and support offered by a captivated population health management system 100 in accordance with an embodiment of the present invention is shown. The current invention utilizes a modular component system that is comprised of, but not limited to: care coordination 102, clinical delivery 104, ancillary services 106, population health 108, Triple Aim metric assessment 110, other services and support 112, etc. Care coordination 102 may include, but is not limited to: scheduling 102a, sick call 102b, telehealth 102c, disease management 102d, care follow-up 102e, correction action plan 102f, etc. Clinical delivery 104 may include, but is not limited to: physical health 104a, behavioral health 104b, oral health 104c, etc. Ancillary services 106 may include, but are not limited to ambulance transport 106a, pharmacy 106b, mammography 106c, physical therapy 106d, etc. Population health 108 includes, but is not limited to: clinical protocols 108a, clinical outcomes 108b, training 108d, patient education 108e, etc. Triple Aim metric assessment 110 may include, but is not limited to: care access 110a, quality management 110b, cost 110c (e.g., utilization review 110d, financial analysis 110e, etc.), metric dashboards 110f, etc. Other services and support 112 may include, but are not limited to: provider recruitment and retention 112a, EMR integration and optimization 112b, etc.

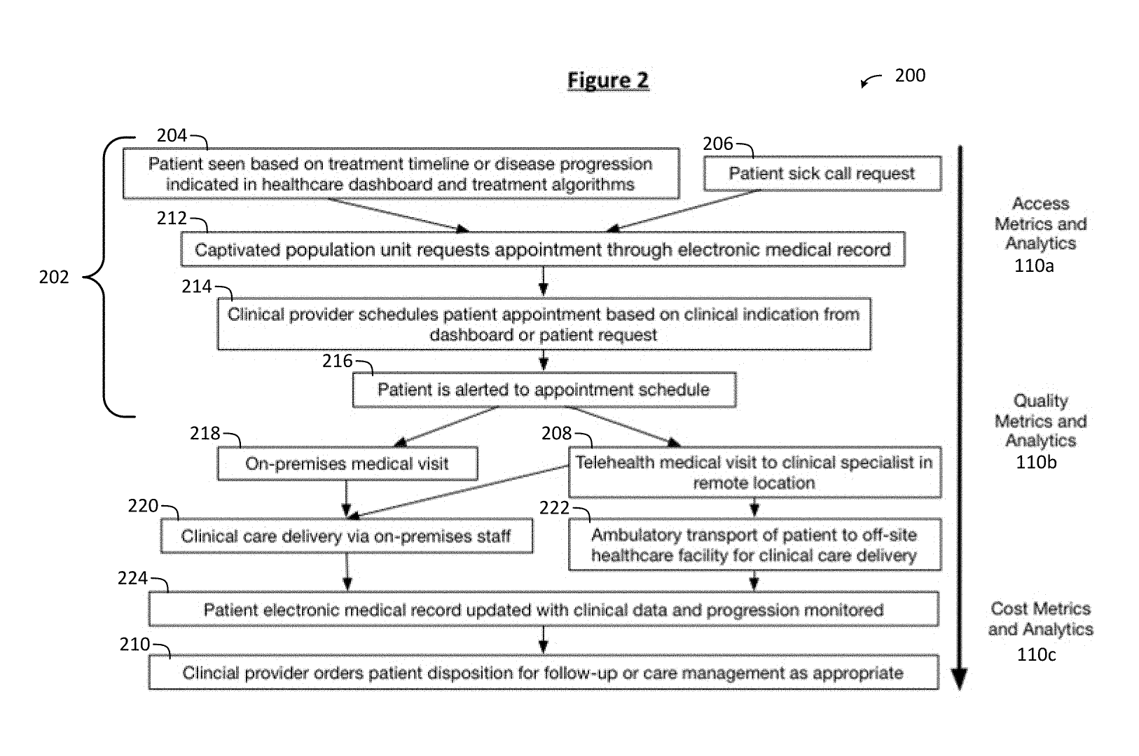

Referring now to FIG. 2, a clinical workflow diagram 200 outlining clinical decision points and analyses metrics in accordance with an embodiment of the present invention is shown. A scheduling component 202 is used for care coordination, which can be initiated from the electronic medical record (EMR) based on the patient being seen on a treatment timeline or disease progression indicated in the healthcare dashboard and treatment algorithms in block 204 or through a requested sick call initiated by the patient in block 206. In a further embodiment of the current invention, the use of telehealth (multi-point audio visual enabled interaction) is used to support the care coordination if the required clinical specialty is not located at the same physical location as the patient in block 208. The care coordination also includes care follow-up and corrective action plan (CAP) development in block 210.

Once the captivated population unit requests the appointment through the EMR in block 212 via blocks 204 or 206, the clinical provider schedules the patient's appointment based on clinical indications from the dashboard or patient initiated requested sick call process in block 214. Once the appointment has been scheduled in block 214, the patient is notified of the appointment schedule in block 216. At this point, if the medical provider is located at the same physical location as the patient as indicated in block 218, the patient receives clinical care via on-premises staff in block 220. If the required medical provider is not located at the same physical location as the patient, telehealth is utilized to conduct a remote medical visit with a medical provider located in a different physical location than the patient in block 208. The remotely conducted medical encounter via telehealth may direct on-premises staff to deliver clinical care to the patient in block 220. If the remotely conducted medical encounter indicates that the patient needs to be further evaluated or examined by the remotely located specialist, the patient, via EMR care coordination and communication, can utilize the ancillary services such as ambulatory transport to be moved off-site to another physical location for further healthcare related services in block 222. All clinical monitoring and interaction during the on-premises staff 220 and the off-site medical encounter 222 are documented and becomes part of the longitudinal patient records embodied in the current invention in block 224. The clinical provider order patient disposition for follow-up and corrective action plan (CAP) development as appropriate in block 210.

FIG. 2 further provides examples of Triple Aim (access 110a, quality 110b, and cost 110c) metrics 110 that can be collected, assessed, compared and reported under the current invention. These metrics can be collected, assessed, compared and reported at a plurality of time points within the progression of the method and system of the current invention. The metrics collected, assessed compared, and reported under this invention are viewable on a plurality of dashboards 110f that can be displayed on an intranet and/or Internet enabled video capable device.

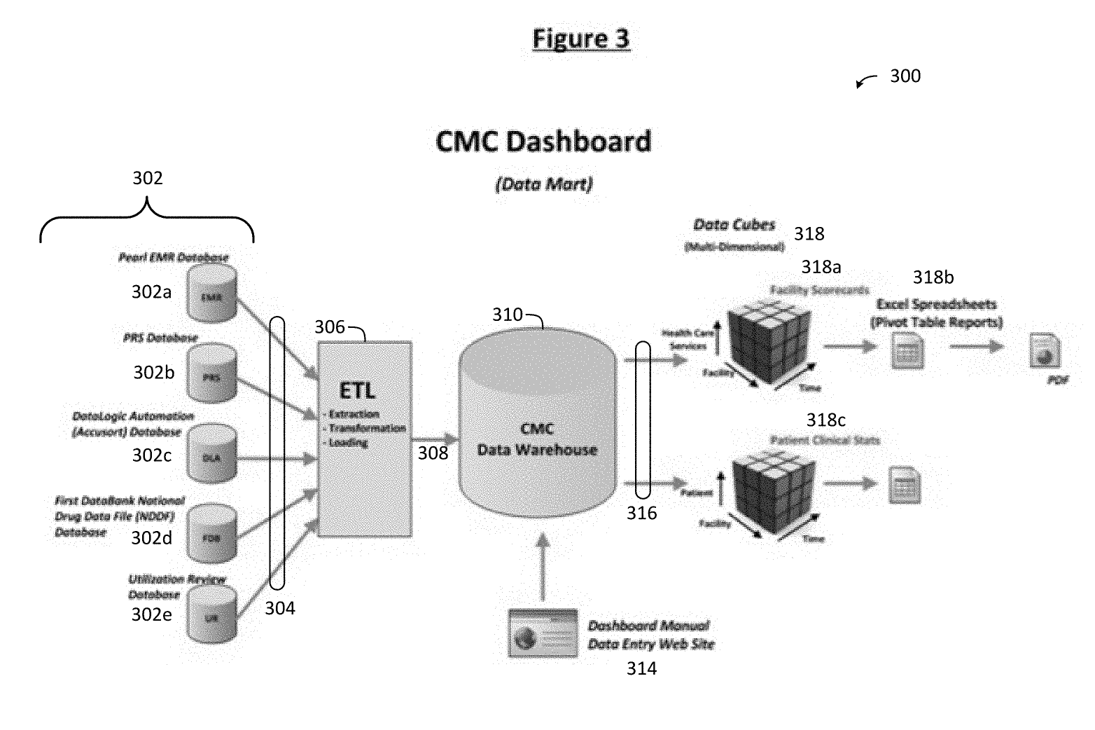

Now referring to FIG. 3, data flow diagram 300 outlining the data transport, data storage, and data reporting (Data Mart) in accordance with an embodiment of the current invention. The schematic outlines the data collection points 302 such as, but not limited to, the Pearl EMR Database (EMR) 302a, Pharmacy Database (PRS) 302b, Datalogic Automation (Accusort) Database (DLA) 302c, First DataBank National Drug Data File (NDDF) Database (FDB) 302d, and Utilization Review Database (UR) 302e. All collected data 304 is brought into the ETL 306 for data extraction, data transformation, and data loading. The extracted, transformed, and loaded data 308 is then moved to the CMC data warehouse 310 for storage. Data 312 may also be manually entered into the data warehouse 310 via data entry dashboards (e.g., web site, etc.) 314. The data 316 located in the data warehouse 310 can then be accessed through a multitude of processes such as, but not limited to, pre-fetch queries, manual queries, or push and pull direct messaging. The data 316 stored in the data warehouse 310 can also be utilized to construct a plurality of multi-dimensional analysis tools 318 including, but not limited to, facility scorecards 318a, Excel spreadsheet (pivot tables) 318b, patient clinical stats 318c, etc. The data 316 stored in the data warehouse 310 can be utilized to build custom dashboards and reports that capture, track, and analyze: Triple Aim (access, quality, and cost) metrics 110, Disease Prevention and Management statistics, Medication Management, Access to Care metrics, Specialty and Hospital Services totals, and Telehealth Encounters. Further, custom dashboards can be constructed to include a multitude of levels that can be aggregated and de-aggregated as needed.

In a preferred embodiment of the current invention, the patient and patient population data can be aggregated for assessment and comparison of larger populations. Patient level data includes, but is not limited to: patient results related to disease prevention and management, disease states and criteria, patient process, lifestyle improvement opportunities, etc. Unit level data includes, but is not limited to: patient lists (patients that make up a unit) related to disease prevention and management, patient and unit identifiers and criteria, unit process improvement opportunities, etc. Facility level data includes, but is not limited to: unit lists related to disease prevention and management, unit and facility identifiers and criteria, facility process improvement opportunities, etc. Regional level data includes, but not limited to: facility lists related to disease prevention and management, facility identifiers and criteria, facility process improvement opportunities, etc. In a further preferred embodiment of the current invention, more complex data aggregation and de-aggregation is provided, allowing greater sorting for disease prevention and management data based on geographical identifiers.

In a preferred embodiment of the current invention, all dashboards and reports are arranged around a plurality of glossaries. Examples of these glossaries are provided below, which include, but are not limited to: Disease Management Glossaries, Access to Care Glossaries, etc. Each glossary is typically comprised of Measure, Target Goal, Definition, Methodology, Corrective Action Threshold, and the HEDIS 2013 Corrective Action Target (Medicaid Standard). The outputs of the dashboard and related spreadsheets and reports are a direct result of the definitions and methodologies used to generate each measure against the target. Disease states include, but are not limited to, the following: Diabetes (HbA1c, LDL, Blood Pressure, nephropathy screening), Hypertension (BP), Coronary artery Disease (LDL), Asthma (prescribed appropriate treatment), etc.

An example of a Disease Management section of the Disease Management Glossary is shown below.

Disease Management Glossary
Disease Management

| | | | | Corrective | HEDIS |
|---|---|---|---|---|---|
| | Target | | | Action | 2013 |
| Measure | Goal | Definition | Methodology | Threshold | (Medicaid) |
| Asthma | 90% | Percentage of patients who were | Denominator: | 81% | 73% |
| | | prescribed the appropriate | Total number of | | |
| | | treatment based on the current | patients who meet | | |
| | | CMC asthma disease | the criteria to be | | |
| | | management guideline at the | included. | | |
| | | time of the report. To be | | | |
| | | included, the patient must meet | | | |
| | | the criteria below: | | | |
| | | 1. The patient must have the | Numerator: | | |
| | | condition mild persistent, | Number of points | | |
| | | moderate persistent, or severe | in the above | | |
| | | persistent asthma for at least 12 | group with active | | |
| | | months Asthma 90″/o prior to | orders for inhaled | | |
| | | the report date. This is based | corticosteroids | | |
| | | upon the first observed date for | | | |
| | | the diagnosis in the EMR (ICD9 | | | |
| | | codes 493.2 \| 493.3 \| 493.4) | | | |
| | | 2. The patient must have been in | | | |
| | | TDCJ custody for at least 12 | | | |
| | | months. This is based upon the | | | |
| | | patient's received date. (The 12 | | | |
| | | month custody period and the 12 | | | |
| | | month condition period must be | | | |
| | | concurrent) | | | |
| Hypertension | 55% | Percentage of hypertension | Denominator: | 50% | 56% |
| (both <60 | | patients whose most recent | Total number of | | |
| and >60) | | blood pressure measurement at | patients who met | | |
| | | the time of the report met both | the criteria to be | | |
| | | the systolic and diastolic | included. | | |
| | | standards for their age group. To | | | |
| | | be included, the patient must | | | |
| | | meet the three criteria below: | | | |
| | | 1. The patient must have a | Numerator: | | |
| | | current diagnosis of | Number of | | |
| | | hypertension which was first | patients in the | | |
| | | observed at least 12 months | above group | | |
| | | prior lo the report date. (All | whose systolic | | |
| | | ICD9 codes starting with 401.) | and diastolic the | | |
| | | This is based upon first | blood pressures | | |
| | | observed dale for the diagnosis | met the standard | | |
| | | in the EMR. | for their age | | |
| | | | group. | | |
| | | 2. The patient must have been in | Patients are sorted | | |
| | | TDCJ custody for at least 12 | by their age | | |
| | | months. This is based upon the | group, and a | | |
| | | patient's received date. (The 12 | calculation is | | |
| | | month custody period and the 12 | conducted for | | |
| | | month condition period must be | each age group. | | |
| | | concurrent) | | | |
| | | 3. The patient must have a | | | |
| | | documented blood pressure in | | | |
| | | the EMR within 12 months of | | | |
| | | the date of the report. | | | |
| | | For hypertension patients <60 | | | |
| | | y/o, the standard for systolic | | | |
| | | blood pressure was ≤140, and | | | |
| | | the standard for diastolic blood | | | |
| | | pressure was ≤90 (less than or | | | |
| | | equal to 140/90). | | | |
| | | For hypertension patients ≥60 | | | |
| | | y/o, the standard for systolic | | | |
| | | blood pressure was ≤150, and | | | |
| | | the standard for diastolic blood | | | |
| | | pressure was ≤90 (less than or | | | |
| | | equal lo 150/90). | | | |
| Coronary | 55% | Percentage of CAD patients | Denominator: | 50% | 41% |
| Artery | | whose most recent low density | Total number of | | |
| Disease | | lipoprotein (LDL) result at the | patients who met | | |
| (CAD) | | time of the report was <100 | the criteria to be | | |
| | | mg/dl. To be included, the | included. | | |
| | | patient must meet the three | | | |
| | | criteria below: | | | |
| | | 1. The patient must have the | Numerator: | | |
| | | condition CAD (defined below) | Number of | | |
| | | for at least 12 months prior to | patients in the | | |
| | | the report date. This is based | above group | | |
| | | upon the first observed date for | whose LDL was | | |
| | | the diagnosis. | ≤100 mg/dl. | | |
| | | CAD defining diagnoses | | | |
| | | include: myocardial infarction | | | |
| | | or who have a diagnosis of | | | |
| | | ischemic vascular disease (IVD) | | | |
| | | (ICD9 codes starting with 410 \| | | | |
| | | 411 \| 412 \| 413 \| 414) | | | |
| | | 2. The patient must have been in | | | |
| | | TDCJ custody for at least 12 | | | |
| | | months. This is based upon the | | | |
| | | patient's received date. (The 12 | | | |
| | | month custody period and the 12 | | | |
| | | month condition period must be | | | |
| | | concurrent) | | | |
| | | 3. The patient must have a | | | |
| | | documented LDL result in the | | | |
| | | EMR within 12 months of the | | | |
| | | date of the report. | | | |
| Diabetes BP | 55% | Percentage of diabetes patients | Denominator: | 50% | 59% |
| | | whose most recent blood | Total number of | | |
| | | pressure was less than or equal | patients who met | | |
| | | to 140/90. Both the systolic and | the criteria to be | | |
| | | diastolic pressure must meet the | included. | | |
| | | standard. To be included, the | | | |
| | | patient must meet the three | | | |
| | | criteria below: | | | |
| | | 1. The patient must have the | Numerator: | | |
| | | condition diabetes (any type) for | Number of | | |
| | | at least 12 months prior to the | patients in the | | |
| | | report date. This is based upon | above group | | |
| | | the first observed date for the | whose BP was | | |
| | | diagnosis in the EMR. | ≤140/90 | | |
| | | 2. The patient must have been in | | | |
| | | TDCJ custody for at least 12 | | | |
| | | months. This is based upon the | | | |
| | | patient's received date. (The 12 | | | |
| | | month custody period and the 12 | | | |
| | | month condition period must be | | | |
| | | concurrent) | | | |
| | | 3. The patient must have a | | | |
| | | documented BP result in the | | | |
| | | EMR within 12 months of the | | | |
| | | date of the report. | | | |
| Nephropathy | 80% | Percentage of diabetes patients | Denominator: | 72% | 78% |
| Screening | | who have either had a | Total number of | | |
| | | documented urine microalbumin | patients who met | | |
| | | result in the EMR or who have | the criteria to be | | |
| | | an active prescription for an | included. | | |
| | | ACE inhibitor or ARB | | | |
| | | medication. To be included, | | | |
| | | the_patient must meet the two | | | |
| | | criteria below: | | | |
| | | 1. The patient must have the | Numerator: | | |
| | | condition diabetes (any type) for | Number of | | |
| | | at least 12 months prior to the | patients in the | | |
| | | report date. This is based upon | above group | | |
| | | the first observed date for the | whose either have | | |
| | | diagnosis in the EMR. | a documented | | |
| | | | urine | | |
| | | | microalbumin | | |
| | | | result in the EMR | | |
| | | | or who have an | | |
| | | | active prescription | | |
| | | | for an ACE | | |
| | | | inhibitor or an | | |
| | | | ARB medication. | | |
| | | 2. The patient must have been in | | | |
| | | TDCJ custody for at least 12 | | | |
| | | months. This is based upon the | | | |
| | | patient's received date. (The 12 | | | |
| | | month custody period and the 12 | | | |
| | | month condition period must be | | | |
| | | concurrent) | | | |
| Diabetes | | Percentage of diabetes patients | | | |
| | | who met the various criteria and | | | |
| | | standards below. (ICD9 codes | | | |
| | | that start with 250.) | | | |
| 1. Diabetes | 45% | Percentage of diabetes patients | Denominator: | 41% | 47% |
| HbA1c | | whose most recent HbA1c result | Total number of | | |
| | | at the time of the report was | patients who met | | |
| | | <8%. To be included, the patient | the criteria to be | | |
| | | must meet the three criteria | included. | | |
| | | below: | | | |
| | | 1. The patient must have the | Numerator: | | |
| | | condition diabetes (any type) for | Number of | | |
| | | at least 12 months prior to the | patients in the | | |
| | | report date. This is based upon | above group | | |
| | | the first observed date for the | whose HbA1c | | |
| | | diagnosis in the EMR. | was <8%. | | |
| | | 2. The patient must have been in | | | |
| | | TDCJ custody for at least 12 | | | |
| | | months. This is based upon the | | | |
| | | patient's received date. (The 12 | | | |
| | | month custody period and the 12 | | | |
| | | month condition period must be | | | |
| | | concurrent) | | | |
| | | 3. The patient must have a | | | |
| | | documented HbA1c result in the | | | |
| | | EMR within 12 months of the | | | |
| | | date of the report. | | | |
| 2. Diabetes | 50% | Percentage of diabetes patients | Denominator: | 45% | 34% |
| LDL | | whose most recent low density | Total number of | | |
| | | lipoprotein (LDL) was <100. To | patients who met | | |
| | | be included, the patient must | the criteria to he | | |
| | | meet the three criteria below: | included. | | |
| | | 1. The patient must have the | Numerator: | | |
| | | condition diabetes (any type) for | Number of | | |
| | | at least 12 months prior to the | patients in the | | |
| | | report date. This is based upon | above group | | |
| | | the first observed date for the | whose LDL was | | |
| | | diagnosis in the EMR. | <100. | | |
| | | 2. The patient must have been in | | | |
| | | TDCJ custody for at least 12 | | | |
| | | months. This is based upon the | | | |
| | | patient's received date. (The 12 | | | |
| | | month custody period and the 12 | | | |
| | | month condition period must be | | | |
| | | concurrent) | | | |
| | | 3. The patient must have a | | | |
| | | documented LDL result in the | | | |
| | | EMR within 12 months of the | | | |
| | | date of the repot. | | | |

An example of a Staffing section of the Disease Management Glossary is shown below.

Disease Management Glossary
Staffing

| | Target | | |
|---|---|---|---|
| Measure | Goal | Definition | Sources |
| Total Actual | N/A | Actual Full Time Equivalent (FTE) number of | Monthly Vacancy Report |
| FTEs | | current FTE staff being utilized through actual | (finance) |
| | | permanent employee filled positions, | |
| | | Overtime and Agency FTEs | |
| Total Authorized | N/A | Authorized FTE number of ongoing positions | Monthly Vacancy Report |
| FTEs | | approved in the FY14 budget | (finance) |
| Vacancy Rate | N/A | Percentage of number of actual FTE numbers | Monthly Vacancy Report |
| | | to number of authorized FTEs | (finance) |
| Medical FTEs | N/A | Number of actual and authorized medical | Monthly Vacancy Report |
| | | staff to include MDs, Dos, PAs and NPs | (finance) |
| Nursing FTEs | N/A | Number of actual and authorized nursing staff | Monthly Vacancy Report |
| | | to include RN's, LVNs, CMAs and PCTs | (finance) |
| Dental FTEs | N/A | Number of actual and authorized dental | Monthly Vacancy Report |
| | | staff to include DDSs, DHs and DAs | (finance) |
| Other Staff | N/A | Number of actual and authorized other | Monthly Vacancy Report |
| FTES | | staff to include CCAs and PMs | (finance) |
| Agency FTEs | N/A | Number of actual agency staff FTEs used | Monthly Vacancy Report |
| | | | (finance) |
| Overtime FTEs | N/A | Number of actual OT FTEs used | Monthly Vacancy Report |
| | | | (finance) |

An example of a Specialty and Hospital Services section of the Disease Management Glossary is shown below.

Disease Management Glossary
Specialty and Hospital Services

| | Target | | |
|---|---|---|---|
| Measure | Goal | Definition | Sources |
| Specialty | N/A | Number of referrals for | HG |
| Care | | specialists consultations | Report |
| Referrals | | submitted and approved | |
| Appointments | N/A | Number of primary and specialty | Operations |
| via | | clinic encounters conducted | Report |
| Tele-health | | via Tele-health | |
| Hospital | N/A | Number of community hospital | UR |
| Admissions | | admissions | Report |
| ER | N/A | Number of community | UR |
| Department | | emergency visits | Report |
| Visits | | | |

An example of a Pharmacy section of the Disease Management Glossary is shown below.

Disease Management Glossary
Pharmacy

| | Target | | |
|---|---|---|---|
| Measure | Goal | Definition | Sources |
| Prescriptions | N/A | Average number of | Pharmacy TDCJ |
| per inmate | | prescriptions dispensed per | Monthly |
| | | inmate per month | Utilization Report |
| Number of | N/A | Percentage of all provider | Pharmacy TDCJ |
| formulary | | generated prescriptions filled | Monthly |
| scripts | | for medications on the | Utilization Report |
| | | formulary | |
| Number of | N/A | Percentage of all provider | Pharmacy TDCJ |
| non- | | generated prescriptions filled | Monthly |
| formulary | | for medications not on | Utilization Report |
| scripts | | the formulary | |
| Reclamation | N/A | Number of dollars reclaimed | Pharmacy TDCJ |
| dollars | | from unused medications | Monthly |
| | | | Utilization Report |

An example of an Access section of the Access to Care Glossary is shown below.

Access to Care Glossary
Access

| | Target | | |
|---|---|---|---|
| Measure | Goal | Definition | Sources |
| Dental | 80% | Each patient who submits a Sick Call Request | Electronic |
| ATC | | for Dental Services will be physically seen by a | Medical |
| | | licensed healthcare worker within 72 hours of | Record |
| | | receipt of the SCR. A Licensed Health Care | |
| | | Professional includes a nurse, medical/dental | |
| | | provider or dental hygienist. | |
| Dental | 80% | Each patient who submits a Sick Call Request | Electronic |
| ATC | | for Dental Services will have chief complaint(s) | Medical |
| | | listed and the date and time the SCR was | Record |
| | | received documented in the medical record. | |
| Dental | 80% | Each patient who has been referred to a dentist | Electronic |
| ATC | | (through nursing dental triage) will be seen by | Medical |
| | | the dentist within 10 working days of receipt of | Record |
| | | the SCR in the Health Services Department. | |
| Nursing | 80% | Each patient who submits a Sick Call Request | Electronic |
| ATC | | for Medical Services (whether NSC or Provider | Medical |
| | | Sick Call) will be physically seen within 72 | Record |
| | | hours. Must be seen by a Licensed Health Care | |
| | | Professional (Nurse [LVN or RN], MD, | |
| | | DO, PA or FNP). | |
| Nursing | 80% | Each patient who submits a Sick Call Request | Electronic |
| ATC | | for Medical Services will have chief | Medical |
| | | complaint(s) documented in the medical record. | Record |
| Medicine | 80% | Each patient who has been referred to a | Electronic |
| ATC | | physician, physician assistant or advanced | Medical |
| | | practice nurse will be seen by the physician, | Record |
| | | physician assistant or advanced practice nurse | |
| | | within 10 working days of receipt of the | |
| | | SCR in the Health Services Department. | |
| Mental | 80% | Each patient on outpatient status who submits a | Electronic |
| Health | | Sick Call Request or is referred for Mental | Medical |
| ATC | | Health Services will be interviewed within 72 | Record |
| | | hours. For triage, the patient must be seen by a | |
| | | Health Care Professional. | |
| Mental | 80% | Each patient who submits a Sick Call Request or | Electronic |
| Health | | is referred for Mental Health Services, will have | Medical |
| ATC | | chief complaint(s) listed and the date and time | Record |
| | | the SCR was received or reason for referral | |
| | | documented in the medical record. | |
| Mental | 80% | Each patient on Outpatient Mental Health status | Electronic |
| Health | | who has been referred to a Qualified Mental | Medical |
| ATC | | Health Professional (QMHP) for further | Record |
| | | evaluation and/or treatment is seen by the | |
| | | QMHP within 10 working days of receipt of the | |
| | | SCR in the Health Services Department. Or | |
| | | notification by Health Care Professional to | |
| | | determine urgency of need as follows: | |
| | | Urgent Mental Health Needs-refer immediately | |
| | | to QMHP, includes but is not limited to risk of | |
| | | suicide or injury to self or others, acute distress | |
| | | or agitation and certain medication side effects. | |
| | | Non-Urgent Mental Health needs-refer to | |
| | | QMHP within 10 working days. | |
| | | Request/Complaint not mental health related- | |
| | | refer patient to appropriate department and | |
| | | follow-up on further requests or referrals. | |

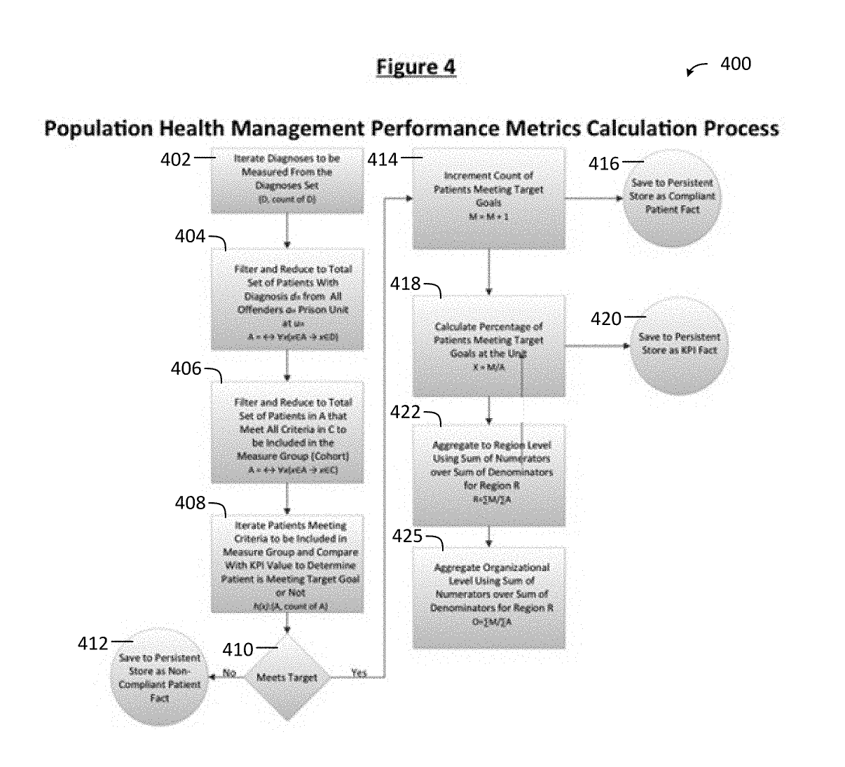

Referring now to FIG. 4, a flow diagram 400 outlining the population health management metrics calculation process in accordance with an embodiment of the present invention is shown. Population health management performance metrics are based off all patients within the captivated population health management system. The population of all patients goes through a series of reduction algorithms based on several factors and criteria before a cohort is created to calculate metrics:
Diagnosis: Diagnosis to be included is configurable and set of diagnoses is iterated to determine the patient population. Diagnoses: D={d 1, d 2, . . . d n}
Key Performance Indicators: A set of Key Performance Indicators is defined. Key Performance Indicators (KPI): K={k 1, k 2, . . . k n}
Criteria: There are several configurable criteria. An example would be that the offender has been incarcerated and in the healthcare system being measured for at least one year. Criteria to be Included in Measure: C={c 1, c 2, . . . c n}
   Patient locations (Patient Units: U={u 1, u 2, . . . u n}) are iterated and the group of all patients is programmatically reduced to the desired Measure Group M which is then iterated for a comparison function h(x) with target values for each and every patient and the count of patients meeting target goals is incremented. The Measure Group is divided by the cohort to yield a percentage meeting targets for a facility.
A=↔∀x(x∈A→x∈D)
A=↔∀x(x∈A→x∈C)
h(x): {A, count of A}
M=M+1
X=M/A
   Data is aggregated to any level defined in the Location Dimension (e.g. R=Region Level) by using sum of numerators over sum of denominators
R=ΣM/ΣA
   KPI Facts, and Patient Facts are created during the iterative process.

Iterate diagnoses to be measured from the diagnosis set {D, count of D} in block 402. Filter and reduce to total set of patients with diagnosis dₙ from all offenders aₙ at prison unit uₙ where A=↔∀x(x∈A→x∈D) in block 404. Filter and reduce to total set of patients in A that meet all criteria in C to be included in the measure group (Cohort) where A=↔∀x(x∈A→x∈C) in block 406. Iterate patients meeting criteria to be included in measure group and compare with Key Performance Indicators (KPI) value to determine patient is meeting target goal or not where h(x): {A, count of A} in block 408. If the target goal is not met, as determined in decision block 410, save to persistent storage as non-compliant patient fact in block 412. If, however, the target goal is met, as determined in decision block 410, increment count of patients meeting target goals where M=M+1 in block 414 and save to persistent storage as compliant patient fact in block 416. Thereafter, calculate percentage of patients meeting target goals at the unit where X=M/A in block 418 and save to persistent storage as KPI fact in block 420. Thereafter, aggregate to region level using sum of numerators over sum of denominators for region R where R=ΣM/ΣA in block 422. Finally, aggregate organizational level using sum of numerators of sum of denominators for region R where O=ΣM/ΣA in block 424.

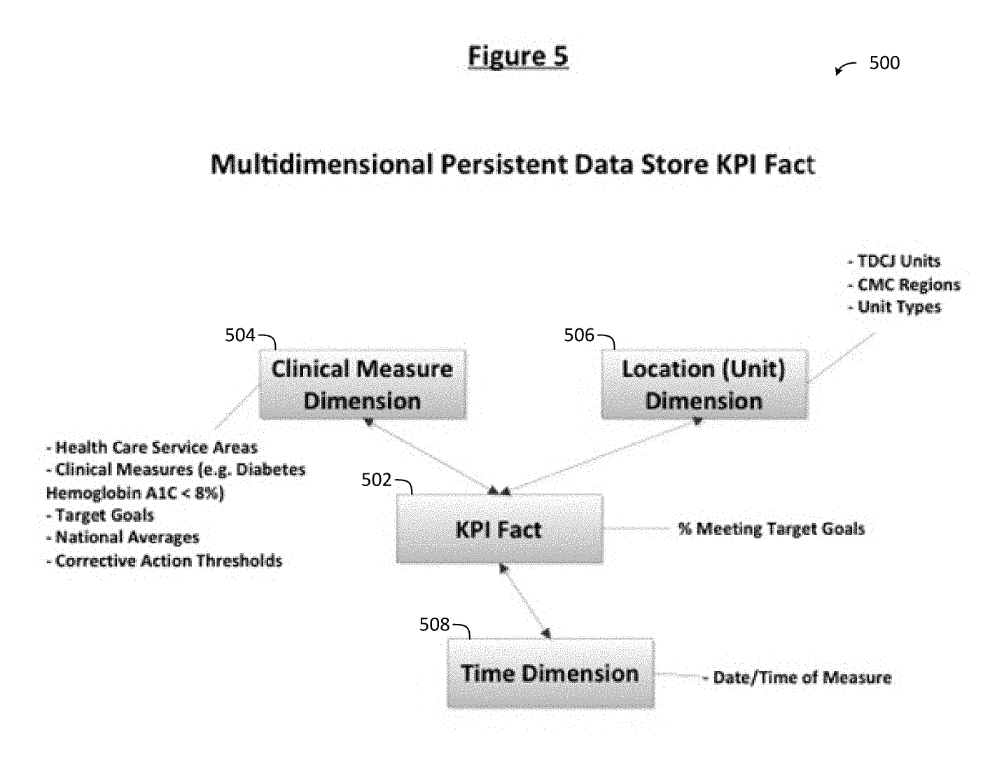

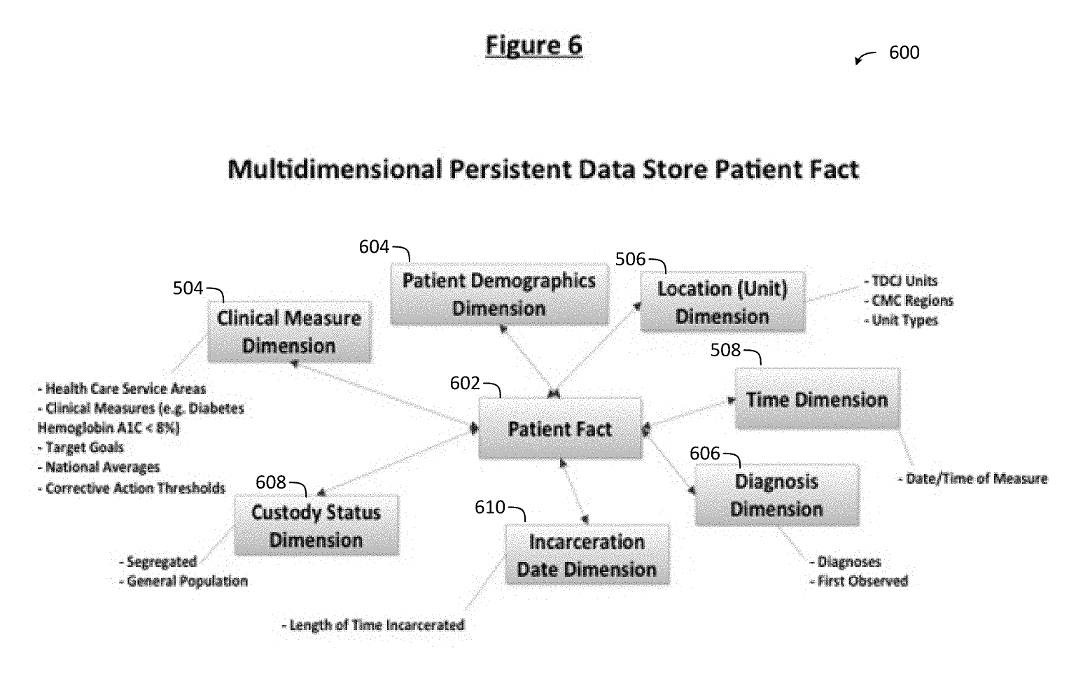

To measure Key Performance Indicators (KPI) for varying and disparate diagnoses, treatment regimens, lab results and other clinical data, a custom-built Online Analytical Processing (OLAP) persistent data store was built to support Multidimensional Analytical (MDA) queries and data mining in dashboard format. Data is stored in the persistent data store as numeric clinical facts categorized by dimensions. Two Facts were defined for Health performance metrics in the captivated healthcare system: a KPI Fact (FIG. 5) and a Patient Fact (FIG. 6).

FIG. 5 is a flow diagram 500 outlining the multidimensional persistent data store KPI fact 502. The KPI Fact 502 (% meeting target goals) provides a multidimensional view of clinical measures 504 in relation to performance indicators for each measure (e.g., health care service areas, clinical measures (e.g., Diabetes Hemoglobin A1C<8%), target goals, national averages, corrective action thresholds, etc.). This view of the metrics allows for comparison of locations 506 (e.g., TDCJ units, CMC regions, unit types, etc.) over time 508 (date/time of measure) and the development of trend analysis.

FIG. 6 is a flow diagram 600 outlining the multidimensional persistent data store patient fact 602. The patient fact provides 602 a multidimensional view of patients included in measures including patient demographics data 604 as well as specific outcomes that were recorded 606 (diagnosis, first observed, etc.), custody status 608 (segregated, general population, etc.), incarceration date 610 (e.g., length of time incarcerated, etc.). This view allows for clinical analysis of outcomes over time 508 (date/time of measure), clinical measurement 504 (e.g., health care service areas, clinical measures (e.g., Diabetes Hemoglobin A1C<8%), target goals, national averages, corrective action thresholds, etc.), locations 506 (e.g., TDCJ units, CMC regions, unit types, etc.), as well as identifying high-risk patient groups and predictive analysis.

In another preferred embodiment of the current invention, non-clinical metrics are collected, assessed, compared, and reported in a plurality of dashboard formats. Non-clinical metrics include, but are not limited to: Access to Care (timeliness), Medical Provider Type, Specialty and Hospital Services (specialty referral volume, telehealth clinic volume, community hospital admissions, community ER visits), Staffing (vacancies by discipline, use of agency staffing, overtime), Pharmacy (formulary and non-formulary volume, prescriptions per patient, reclamation dollars), etc.

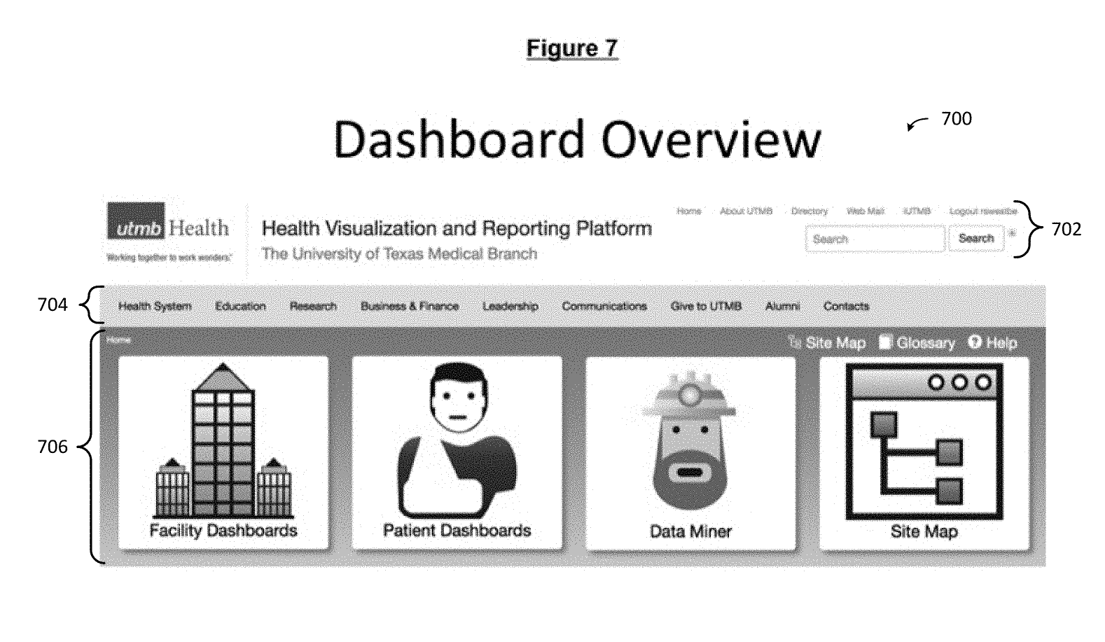

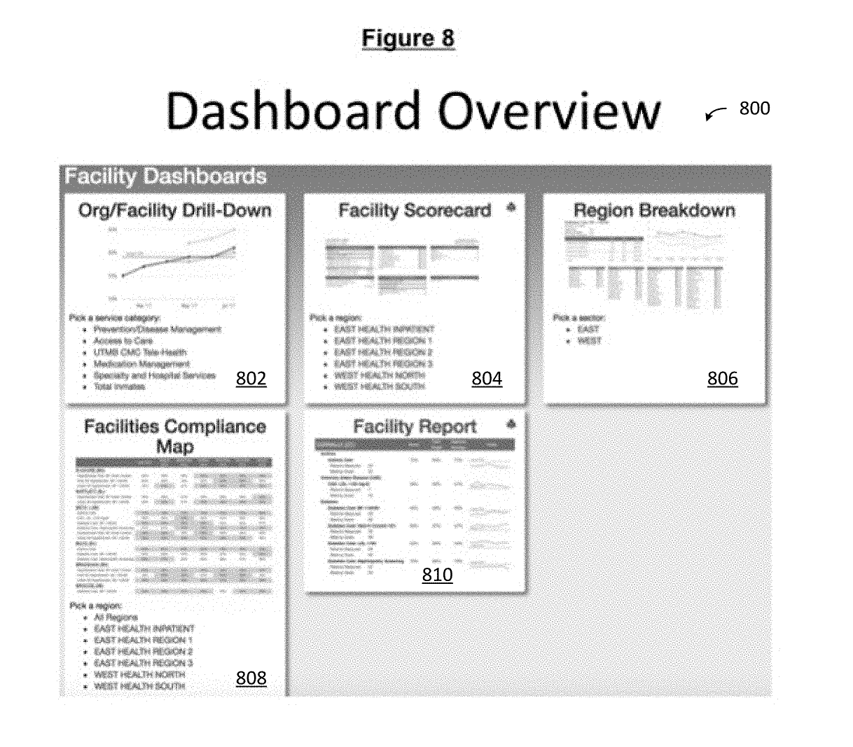

FIGS. 7 and 8 provide example dashboards of an embodiment of the current invention. In FIG. 7, the dashboard 700 includes a first set of links 702 (e.g., Home, About UTMB, Directory, Web Mail, UTMB, Logout, Search, etc.), a second set of links 702 (e.g., Health System, Education, Research, Business & Finance, Leadership, Communications, Give to UTMB, Alumni, Contacts, etc.), and a third set of links (e.g., Home, Facility Dashboards, Patient Dashboards, Data Miner, Site Map, Glossary, Help, etc.). Links 702, 704, 706 are configured to allow users to access internal and/or external functions, information, resources, software, etc. from the dashboard 700. Links 702, 704, 706 can be buttons, tabs, text, icons, graphics, etc. that are single items or part of a navigation bar, menu or other configuration. Note that the current invention is not limited to the links 702, 704, 706 shown or their orientation or configuration. In FIG. 8, an example of Facility Dashboards 800 includes Org/Facility Drill Down 802, Facility Scorecard 804, Region Breakdown 806, Facilities Compliance Map 808 and Facility Report 810. These dashboards display desired information and analytics in graphs, tables, lists and text. The information can be changed, viewed or updated using various functional links or buttons. Note that the current invention is not limited to the links 702, 704, 706 shown or their orientation or configuration.

The current invention provides a Quality Systems and Risk Management program to ensure data integrity, data accuracy, date timeliness. In a preferred embodiment of the current invention, the Quality Systems and Risk Management program employs Lean-Six-Sigma Green Belt and/or Black Belt certified staff to provide quality assessments included, but not limited to: Facility Quality Improvement/Quality Management, Support Services Quality Improvement/Quality Management, Peer Review, Morbidity/Mortality Review, Clinical Quality Review, Pharmacy and Therapeutic Review, Incident Reports, Corrective Action, Root Cause Analysis, etc.

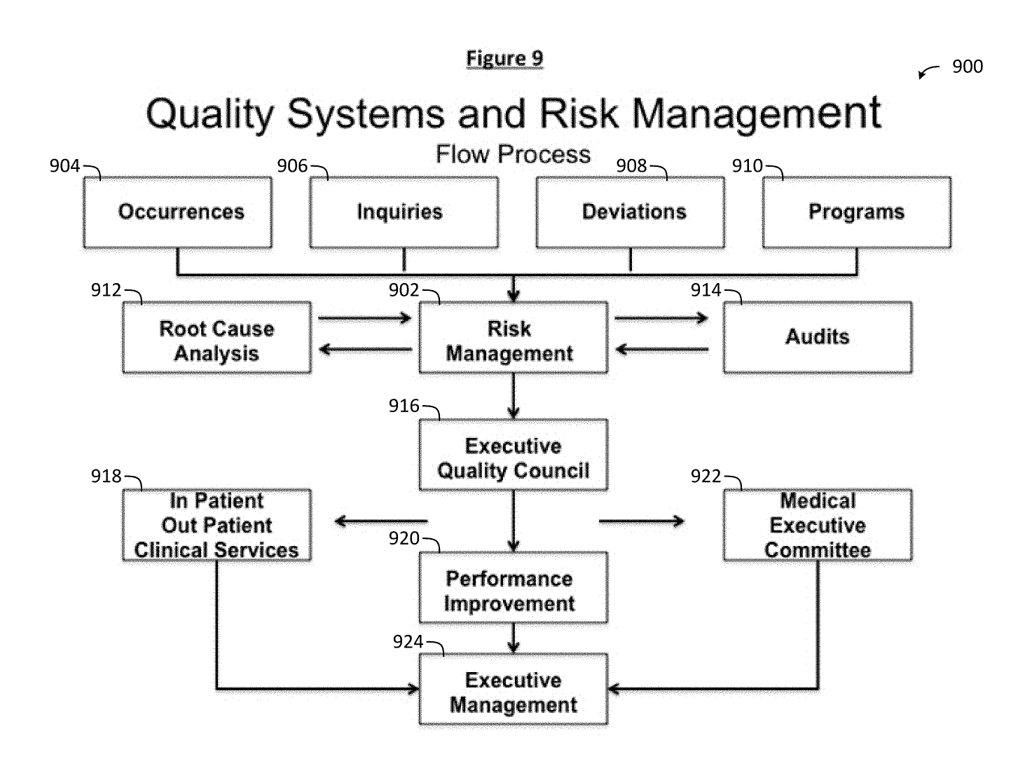

For example, FIG. 9 is a flow diagram of the Quality Systems and Risk Management processes 900 according to an embodiment of the current invention. The risk management process 902 receives data from occurrences 904, inquiries 906, deviations 908 and programs 910. The risk management process 902 provides data to and receives data from root cause analysis 912 and audits 914. The executive quality council 916 uses the risk management process 902 to provide data and recommendations to in patient/out patient clinical services 918, performance improvement 920 and medical executive committee 922, which in turn provide data and recommendations to executive management 924.

Prevention and Disease Management goals include a Corrective Action Threshold (CAT). Any unit that drops below the CAT on a Dashboard Report is required to initiate a Corrective Action Process that includes the following: a Quality Improvement/Quality Management meeting to discuss corrective actions, a Corrective Action Plan prepared by Unit Managers and approved by Regional Managers, Follow Up Tracking for six (6) months.

At the end of the Follow Up Tracking period, if a unit is still below the CAT, the unit must repeat the process: conduct a Quality Improvement/Quality Management Meeting to discuss corrective actions, submit a second Corrective Action Plan approved by Regional Managers, submit to a further six (6) months of Follow Up Tracking at the end of the second Follow Up Tracking period, if a unit is still below the CAT, the unit is referred to the Executive Quality Council for action.

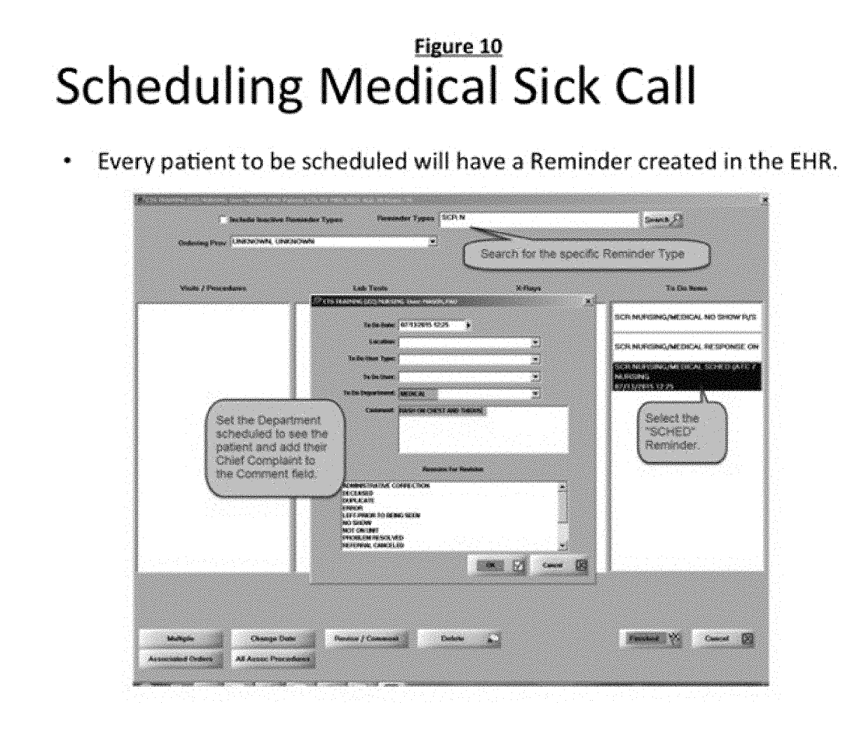

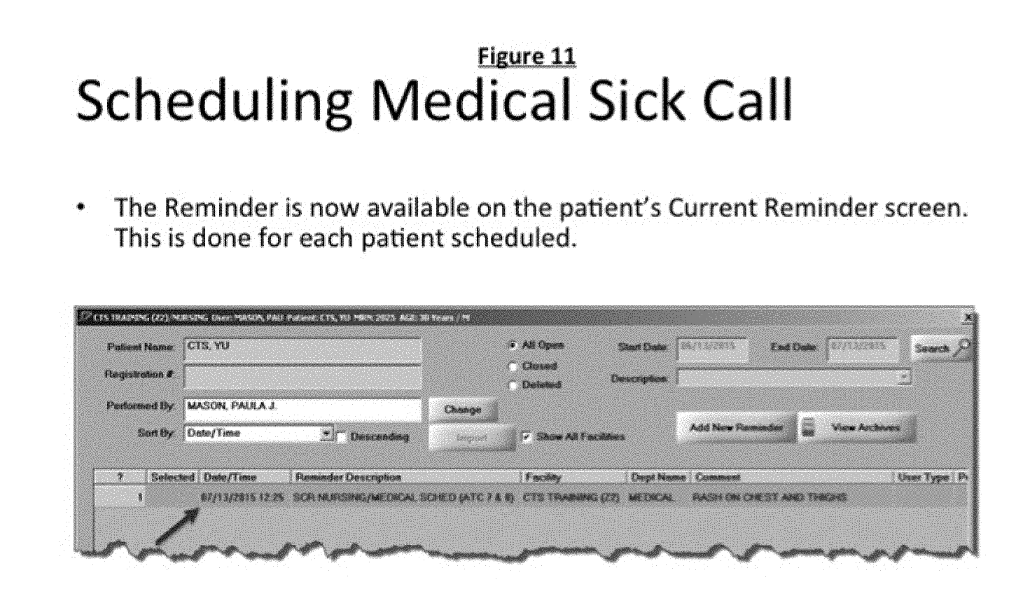

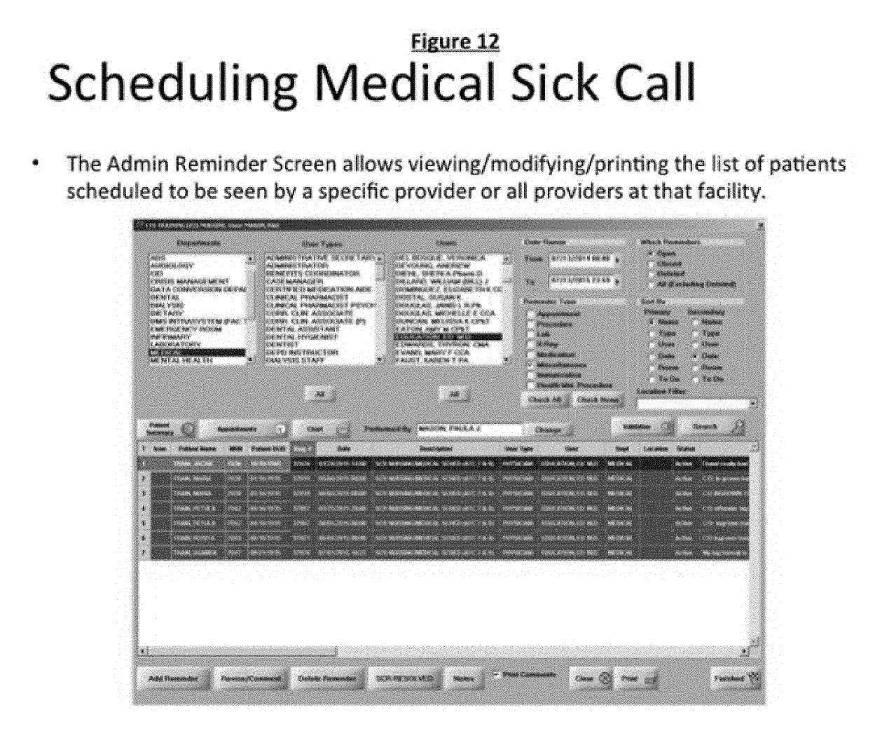

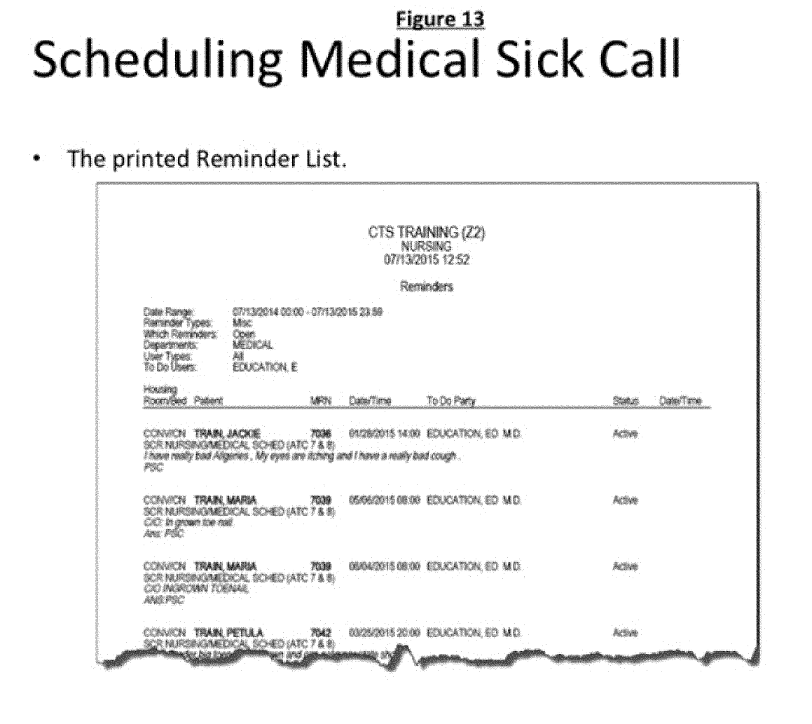

A non-limiting example of the system will now be described. A Diboll Facility patient is feeling dizzy and decides to request sick call at his unit location. The patient's sick call request is documented using the process and interfaces provided in FIG. 10-13. FIG. 10 provides that every patient to be scheduled will have a reminder created in the EMR. FIG. 11 provides that reminder is now available on the patient's Current Reminder Screen. FIG. 12 provides the Admin Reminder Screen that allows viewing, modifying, and printing the list of patients scheduled to be seen by a specific provider or all providers at that facility. FIG. 13 is an example of the printed Reminder List that is provided to the patient for their records.

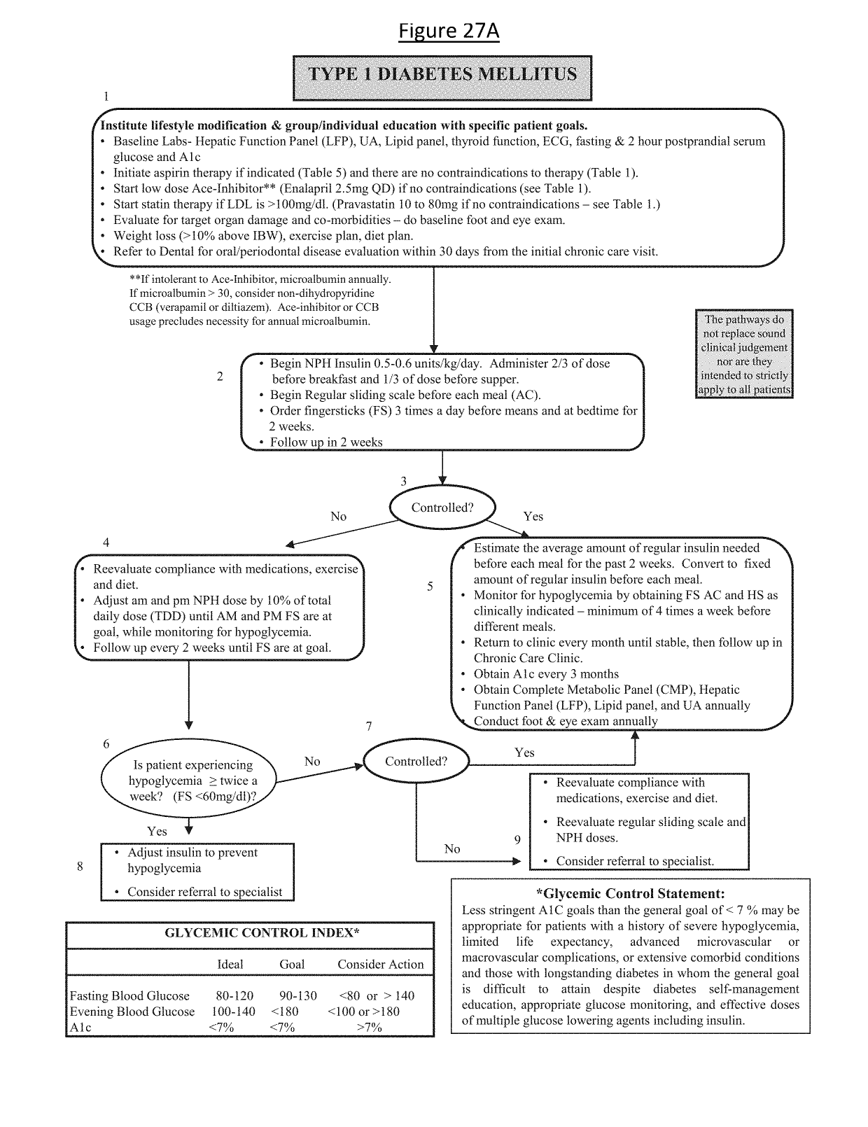

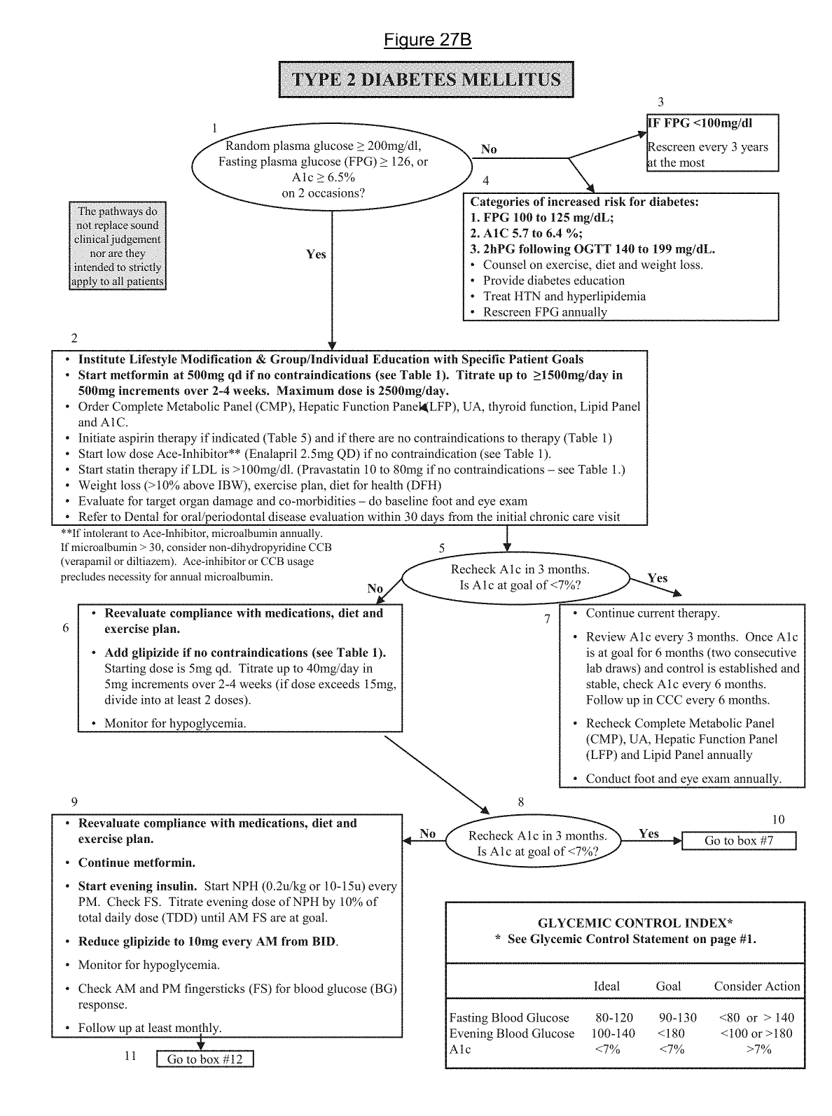

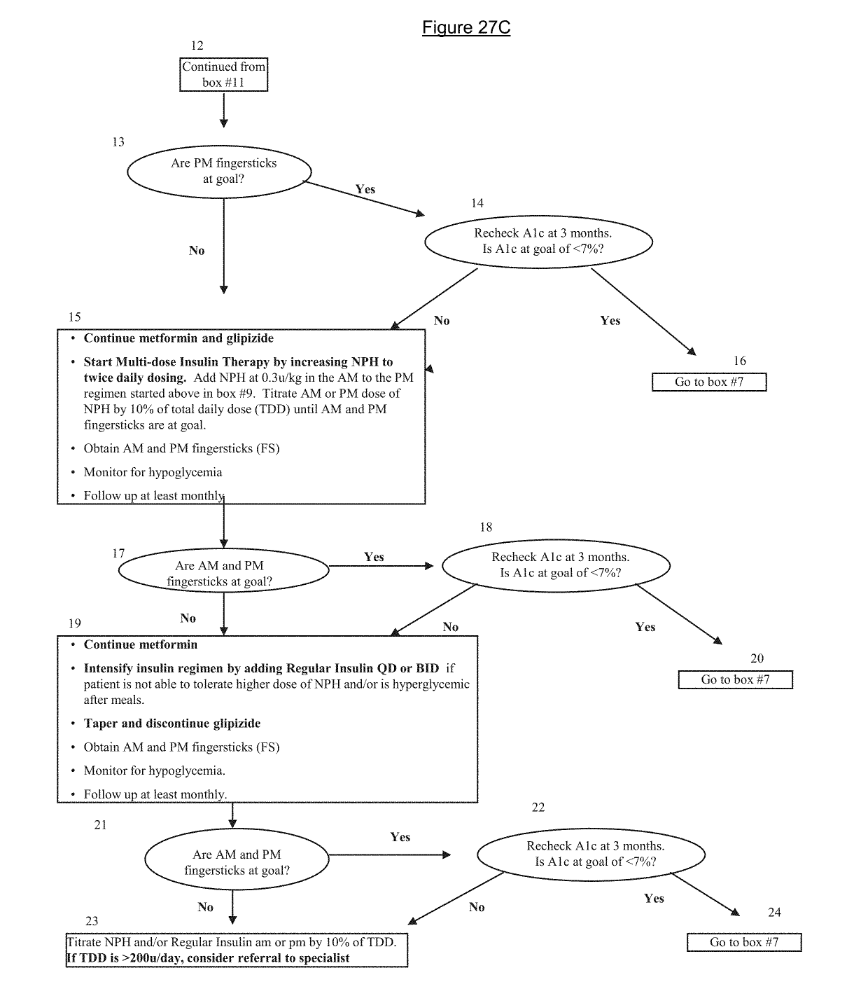

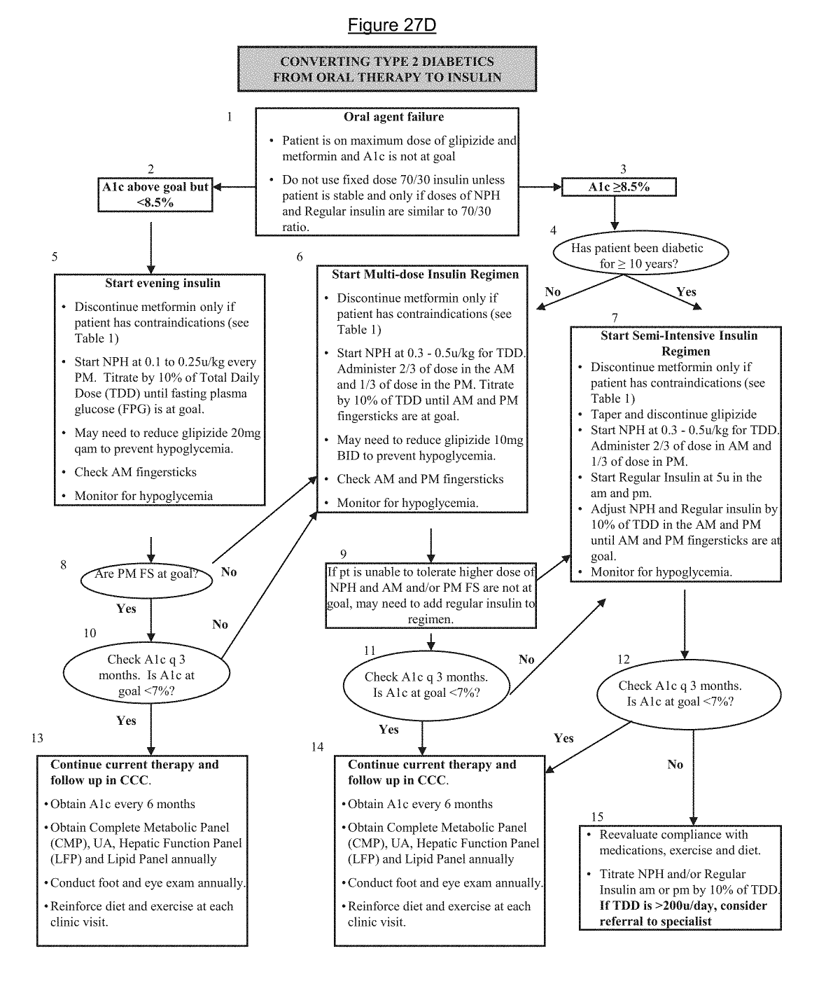

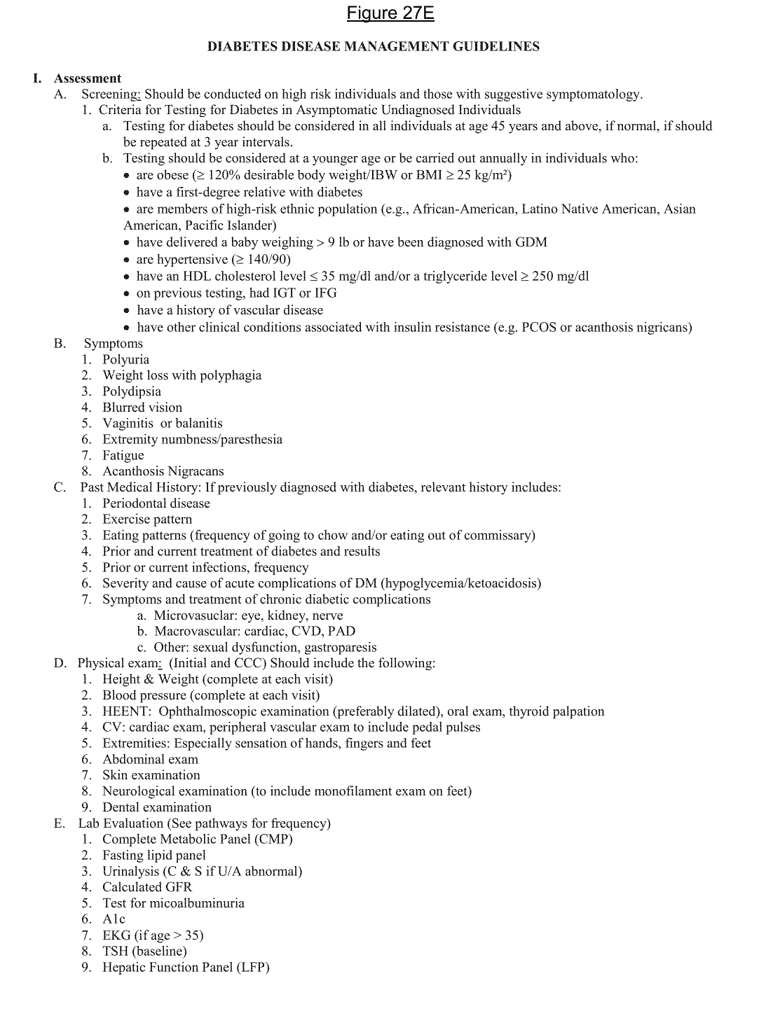

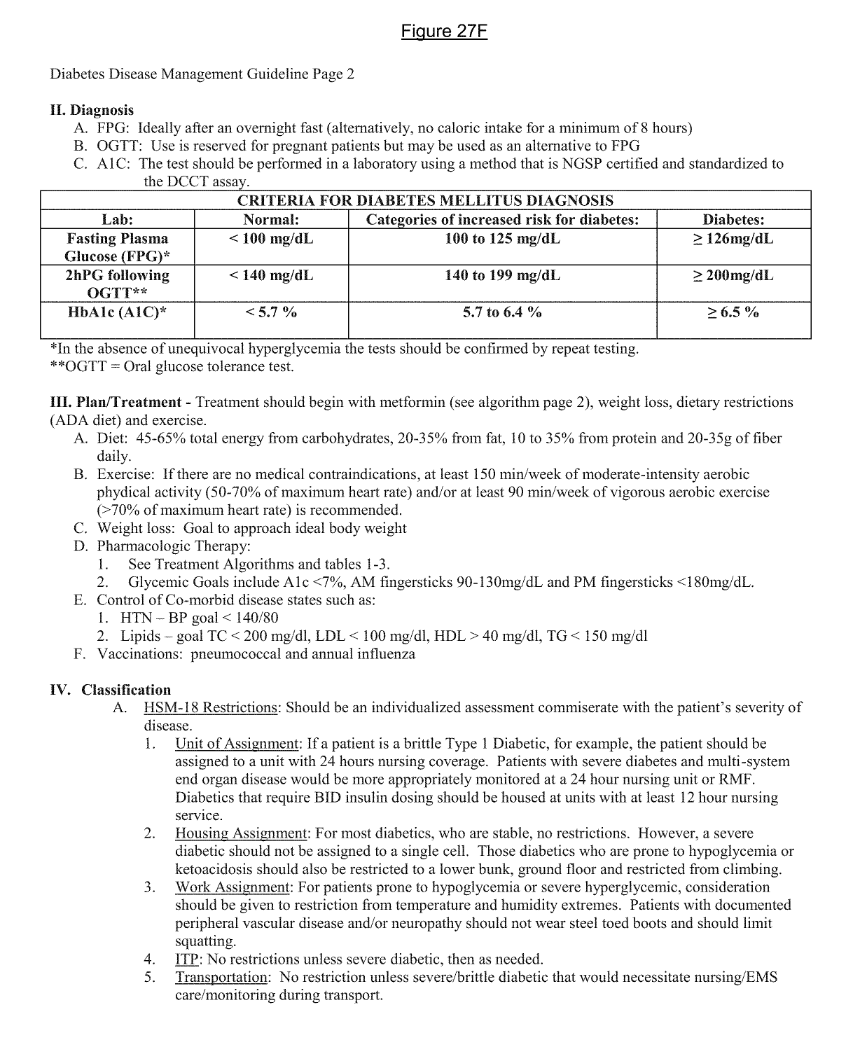

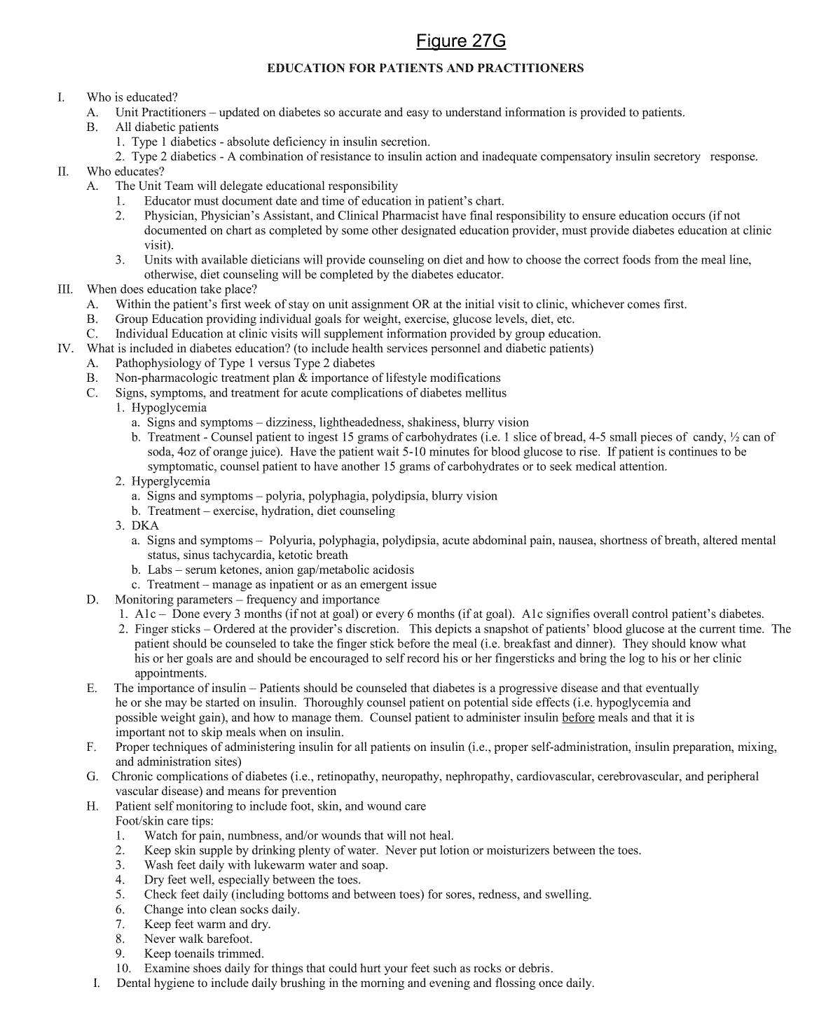

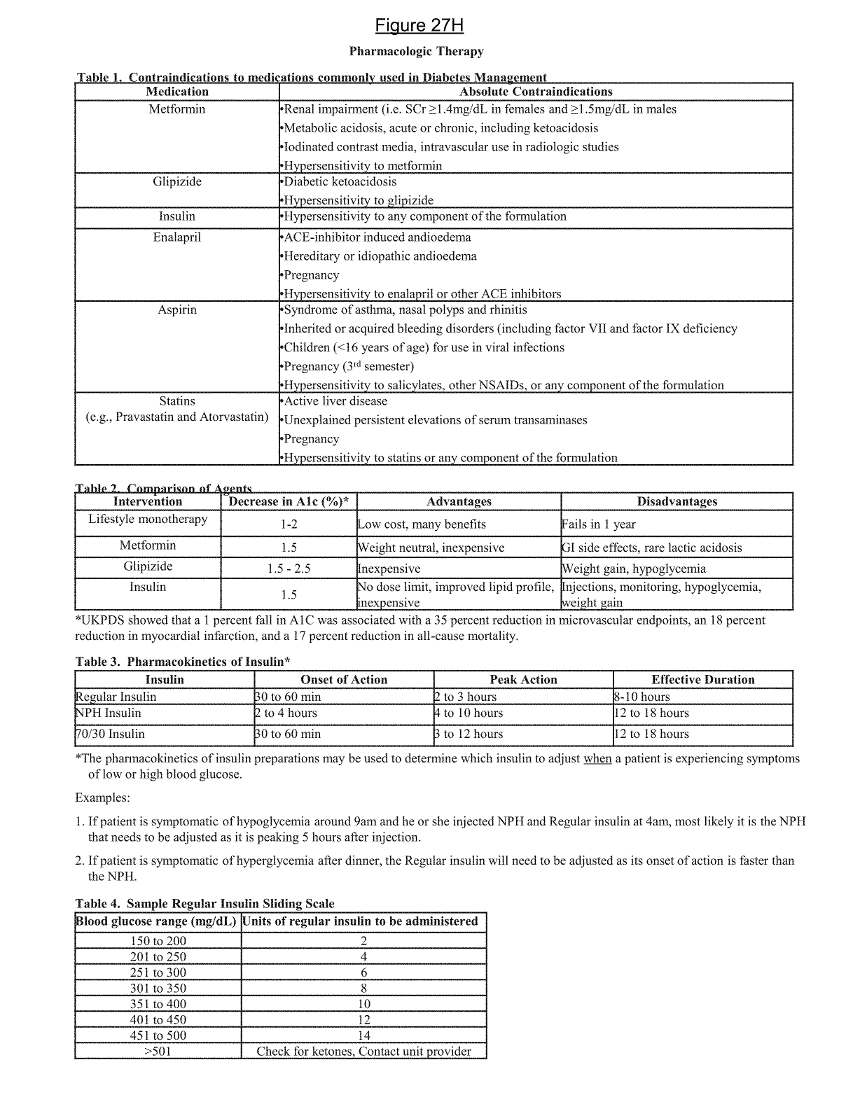

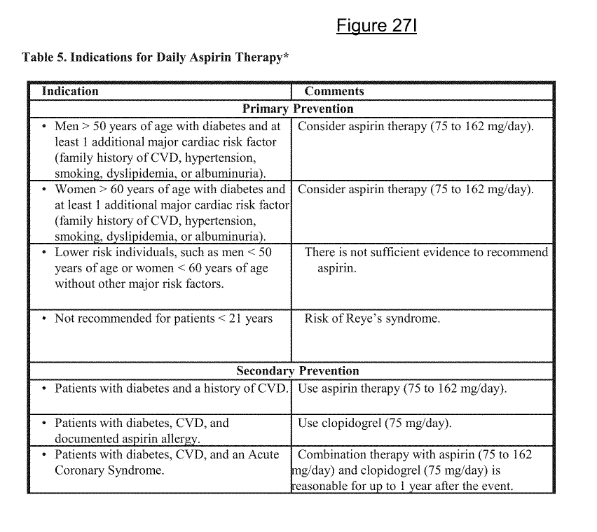

After the patient's appointment has been scheduled, an onsite medical provider performs the clinical encounter with the patient. The local medical provider is not specialized in the area of diabetes mellitus and informs the patient that he will need to be seen by a specialist via telehealth. An offsite specialist provides the patient telehealth specialty care consultation. The specialist utilizes the established diabetes mellitus clinical protocol provided in FIGS. 27A-271. After the telehealth specialty consult, the onsite medical provider can provide the direct clinical care prescribed by the telehealth specialty provider. The medication management system embodied in the current invention is used to track the delivery and compliance of the prescribed medication.

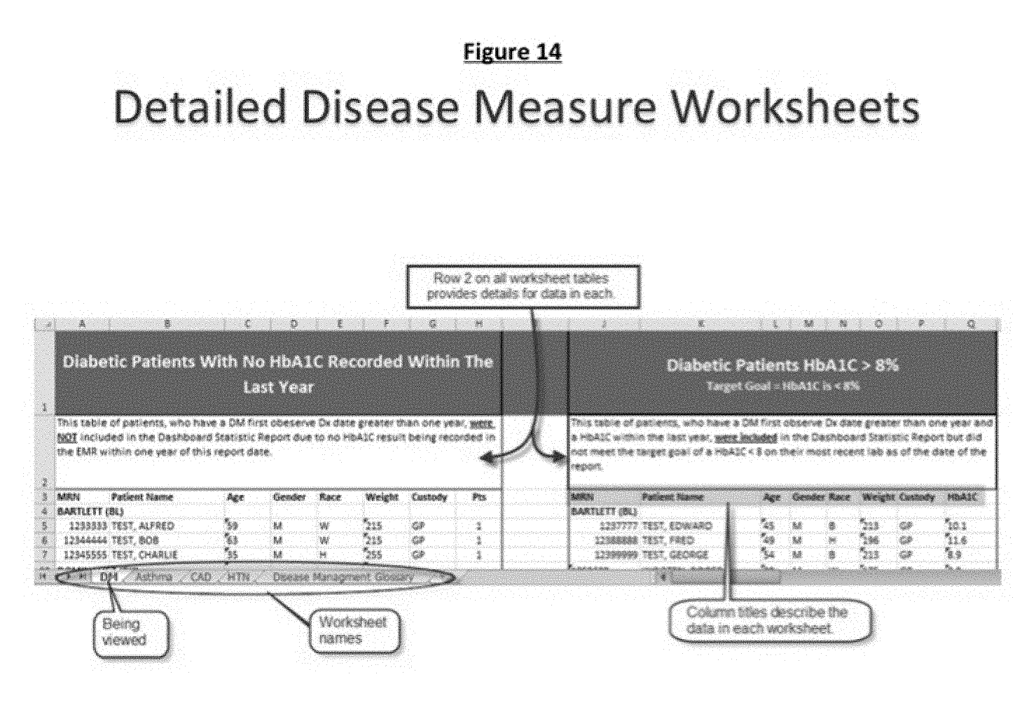

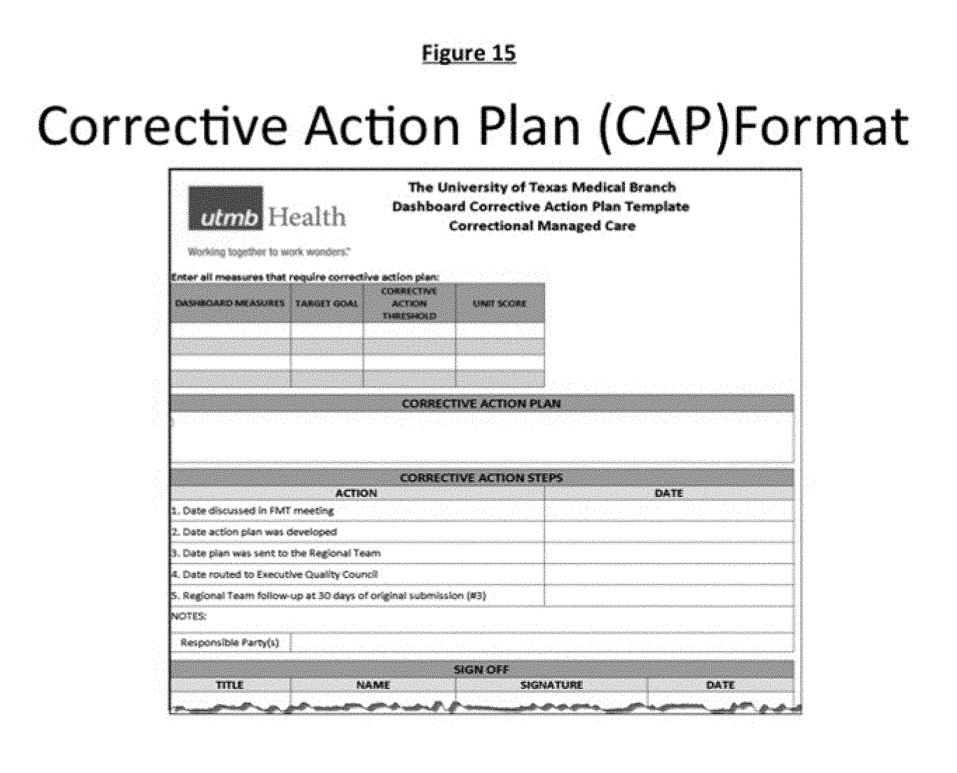

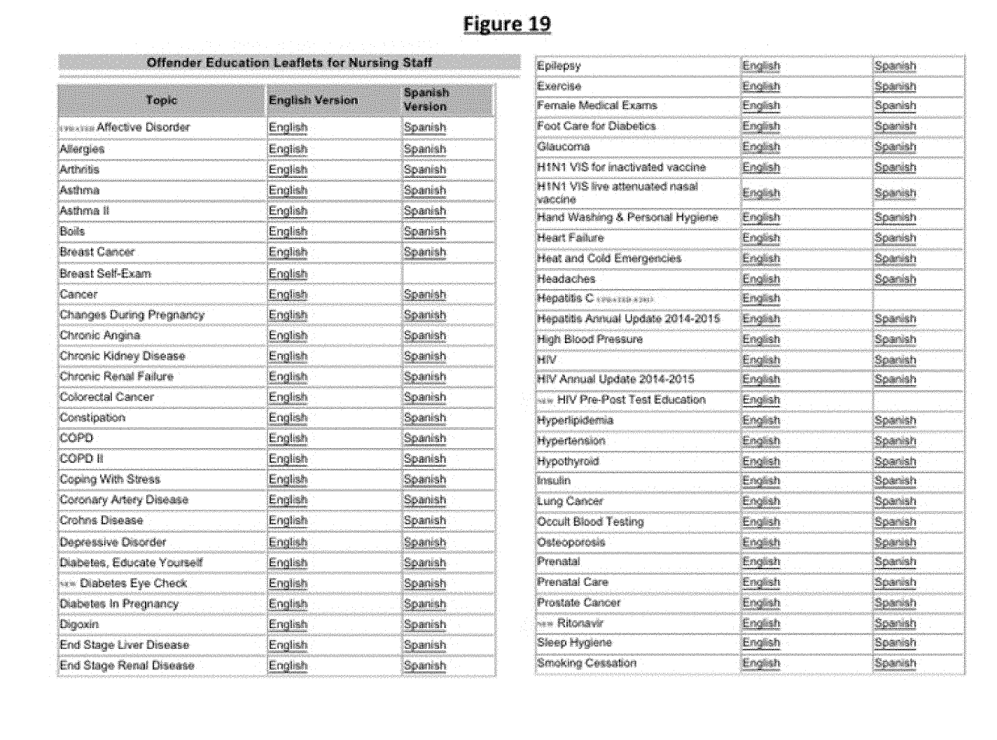

The test results and medication, along with other clinical and non-clinical information become part of the patient's longitudinal patient record within the electronic medical record (EMR). The data is presented in a plurality of dashboards outlined and demonstrated in the current invention. FIG. 14 is an annotated screen shot of a detailed Disease Measurement Work dashboard for the patient based on two diabetic criteria: 1) No HbA1C recorded within the last year and 2) HbAiC>8% patient level dashboard. The individual patient dashboards display patient identifiers such as, but not limited to: Medical Record Number (MRN), Patient Name, Age, Gender, Race, Weight, Custody, etc. Based on the individual patient's medical results, indicated on the dashboards, a corrective action plan could be customized for the patient to work towards reaching clinical care criteria that fall within acceptable targets and overall system goals as illustrated in FIG. 15. FIG. 19 illustrates patient education materials that can assist the patient to achieve the target goals.

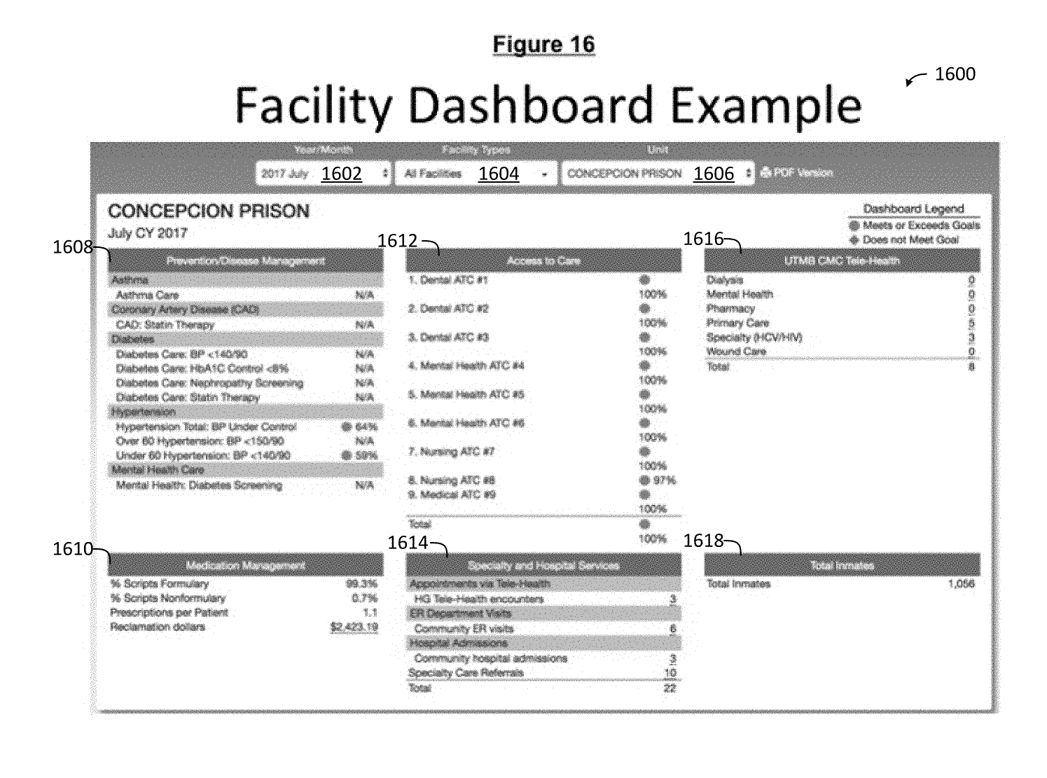

The clinical and non-clinical data collected and reported on the individual patient basis, can be aggregated to other dashboards to better and more accurately demonstrate the assessment of a larger health population such as a unit, facility, region, or state. FIG. 16 provides an illustration of the Concepcion Prison dashboard 1600, made up of aggregate data collected from individual patients at the Concepcion Prison. The dashboard 1600 captures data based on a time frame 1602, a facility type 1604 and/or unit 1606. Other criteria can be used. The dashboard 1600 captures: Prevention and Disease Management metrics 1608 of four major disease states (Asthma, Diabetes, Hypertension, and Coronary Artery Disease (CAD)) along with a multitude of sub categories of each disease state; Medication Management metrics 1610 including reclamation dollars; Access to Care metrics 1612 including Dental, Mental, Physical (Nursing and Medical) care; Specialty and Hospital Services metrics 1614 including community ER visits, community hospital admissions, and specialty care referrals; UTMB CMC Telehealth encounters 1616 by modality; and Total Inmates 1618. The Prevention and Disease Management metrics 1608 and Access to Care metrics 1612 display the percentage as well as a visual performance indicator (e.g., a green light if the metric is greater than or equal to the goal, a red light if the metric is less than the goal). Other metrics and types of visual performance indicators can be used.

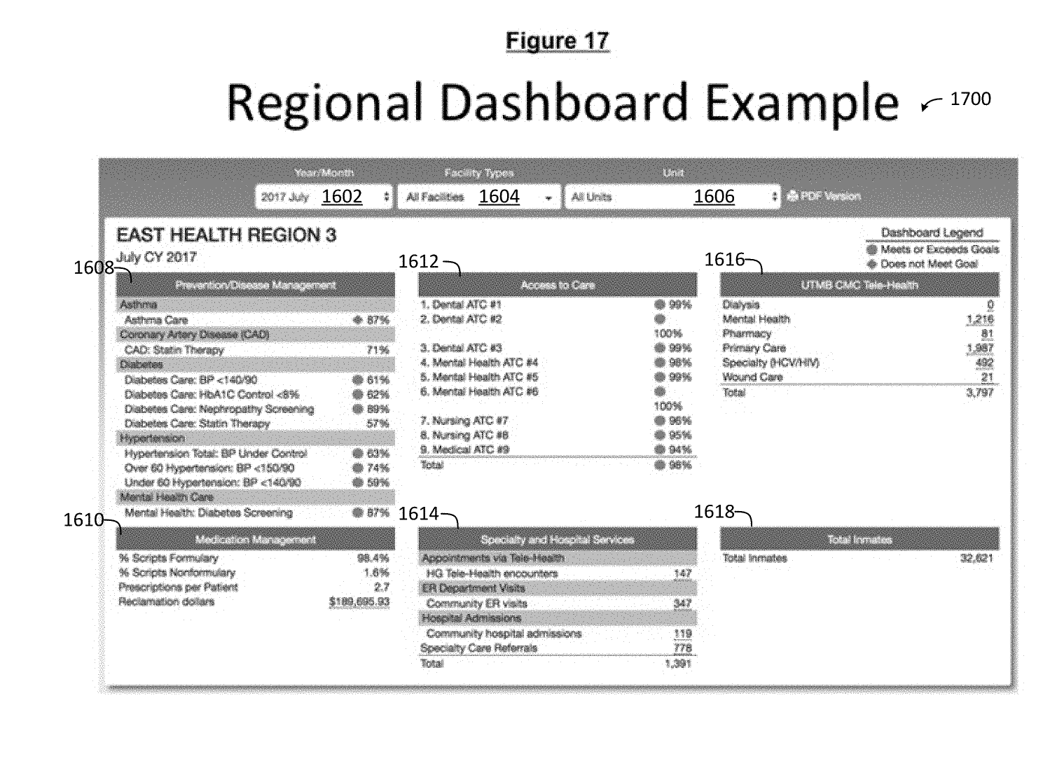

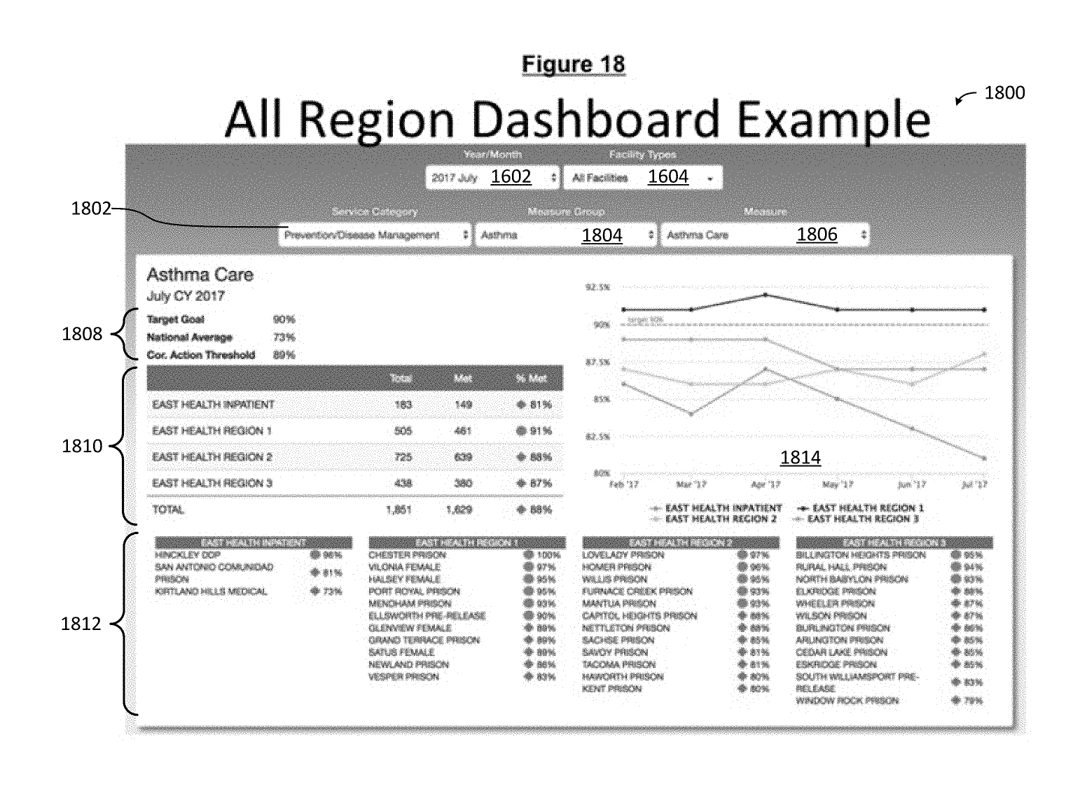

FIGS. 17 and 18 provide illustrations of other stratified dashboards that are a further aggregation of data. FIG. 17 illustrates the Region level dashboard 1700 and associated metrics similar to those shown in FIG. 16. FIG. 18 illustrates the All Region level dashboard 1800 and associated metrics. The dashboard 1800 captures data based on a time frame 1602, a facility type 1604, service category 1802, measure group 1804 and/or measure 1806. Other criteria can be used. The dashboard 1800 captures: target goal, national average and corrective action threshold 1808; metrics 1810 for each region; unit metrics 1812 for each region; and a graph 1814 displaying the metrics for each region over time. The collection, assessment, comparison, and recording of the patient's data become part of the complete population management system that can be used to achieve the Triple Aim 110 (access, quality, and cost).

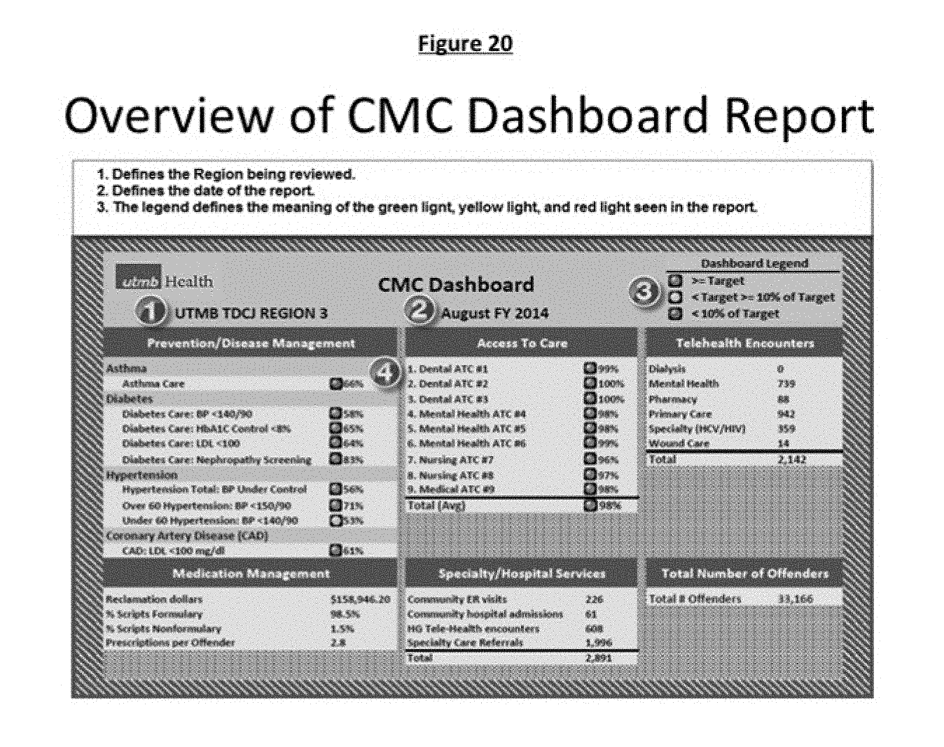

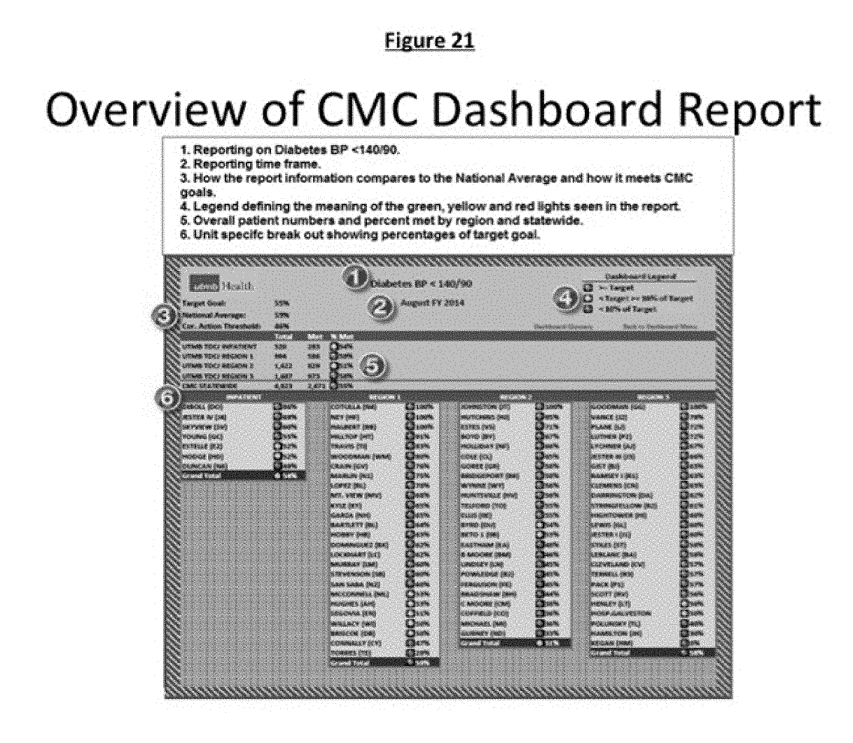

FIGS. 20 and 21 provide example dashboards of another embodiment of the current invention. In FIG. 20, reference 1 identifies the geographical region of the integrated population health management method and system. Reference 2 identifies the time frame. Reference 3 identifies the dashboard target summary. Reference 4 identifies the Prevention and Disease Management results, Medication Management results, Access to Care results, Specialty and Hospital Services results, Telehealth Encounters results, and Total Number of Offenders results.

In FIG. 21, Reference 1 identifies the disease state. Reference 2 identifies the timeframe. Reference 3 identifies the Target Goal, National Average, and Corrective Action Threshold. Reference 4 identifies the dashboard target summary. Reference 5 identifies the summary aggregate Total, Met, and % Met. Reference 6 represents the individual facility metrics that make up the aggregate region metrics shown.

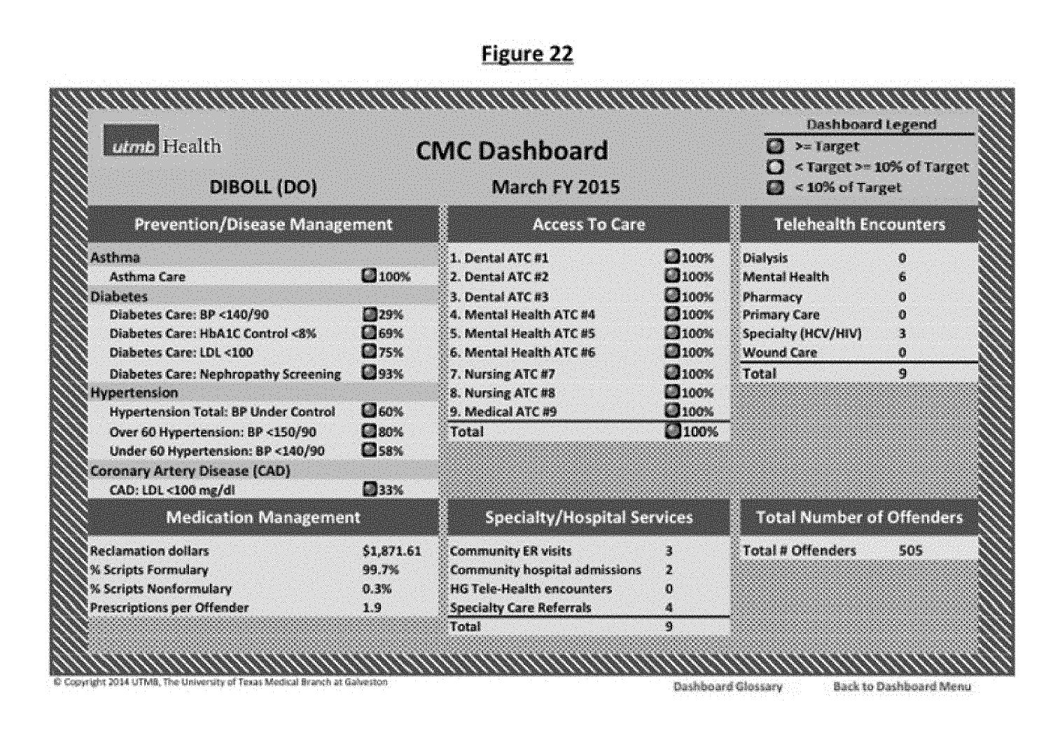

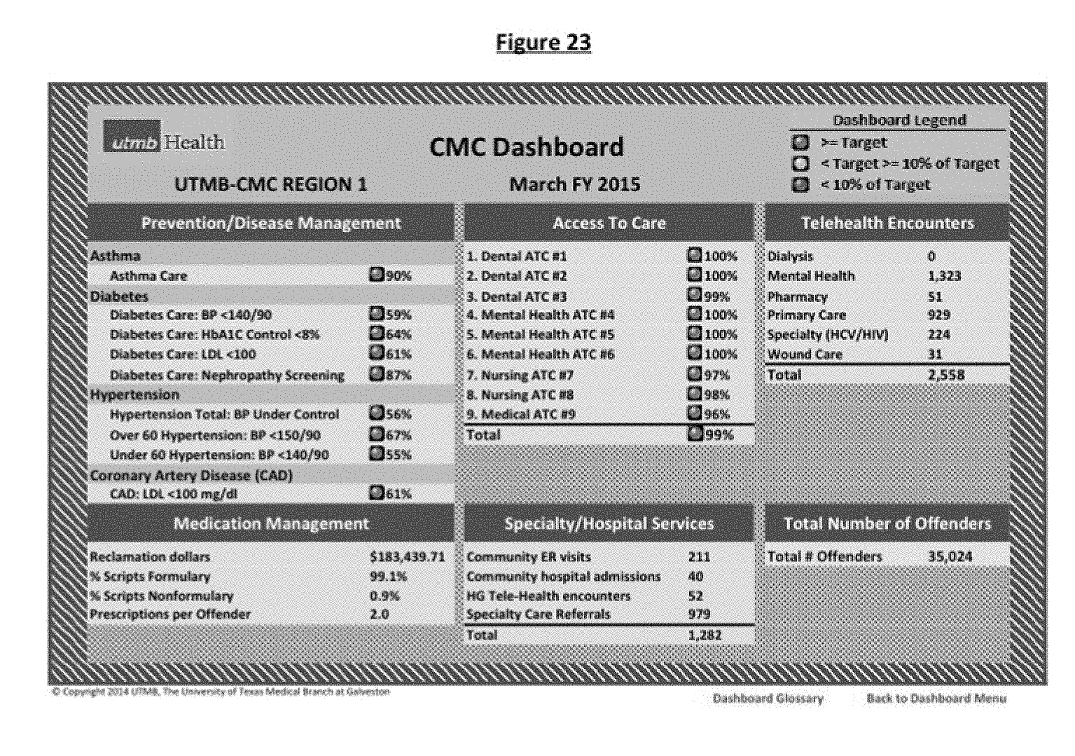

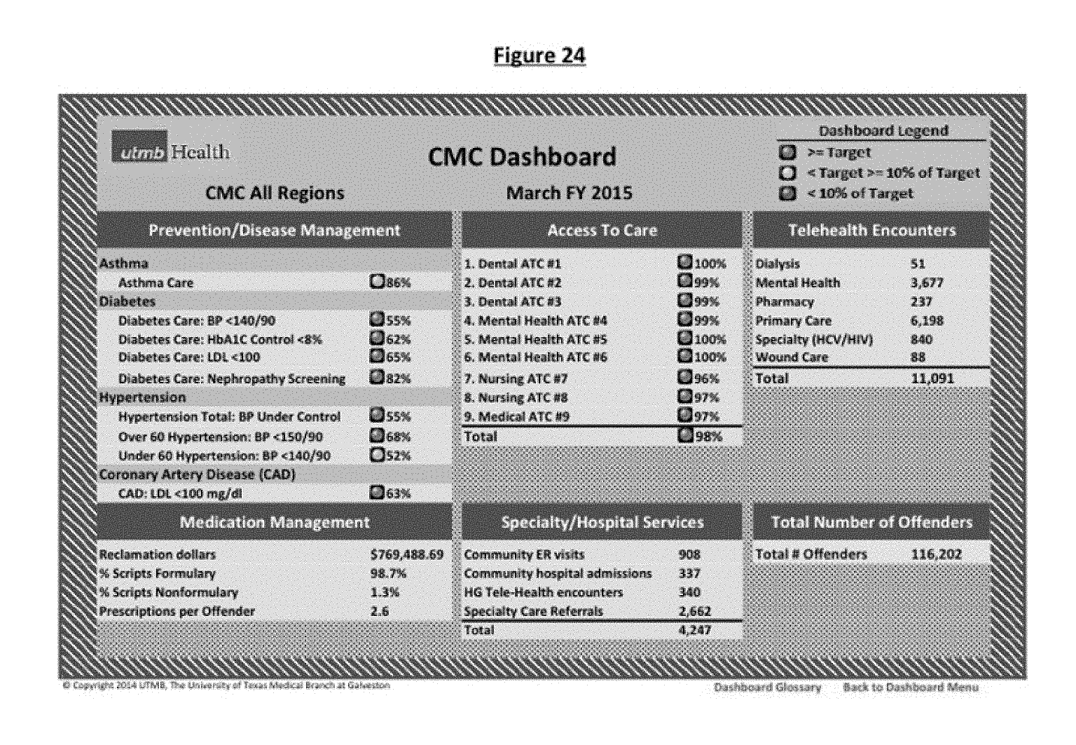

The clinical and non-clinical data collected and reported on the individual patient basis, can be aggregated to other dashboards to better and more accurately demonstrate the assessment of a larger health population such as a unit, facility, region, or state. FIG. 22 provides an illustration of the Diboll Facility dashboard, made up of aggregate data collected from individual patients at the Diboll Facility. The dashboard captures: Prevention and Disease Management metrics of four major disease states (Asthma, Diabetes, Hypertension, and Coronary Artery Disease (CAD)) along with a multitude of sub categories of each disease state; Medication Management metrics including reclamation dollars; Access to Care metrics including Dental, Mental, Physical (Nursing and Medical) care. Specialty and Hospital Services metrics including community ER visits, community hospital admissions, and specialty care referrals; Telehealth Encounter by modality, and Total Number of Offenders. The Prevention and Disease Management metrics and Access to Care metrics display the percentage as well as a visual performance indicator (e.g., a green light if the metric is greater than or equal to the target, a yellow light if the metric is less than the target and greater than or equal to 10% of the target, and a red light if the metric is less than 10% of the target). Other types of visual performance indicators can be used. FIGS. 23 and 24 provide illustrations of other stratified dashboards that are a further aggregation of data. FIG. 23 illustrates the Region level dashboard and associated metrics. FIG. 24 illustrates the All Region level dashboard and associated metrics. The collection, assessment, comparison, and recording of the patient's data become part of the complete population management system that can be used to achieve the Triple Aim 110 (access, quality, and cost).

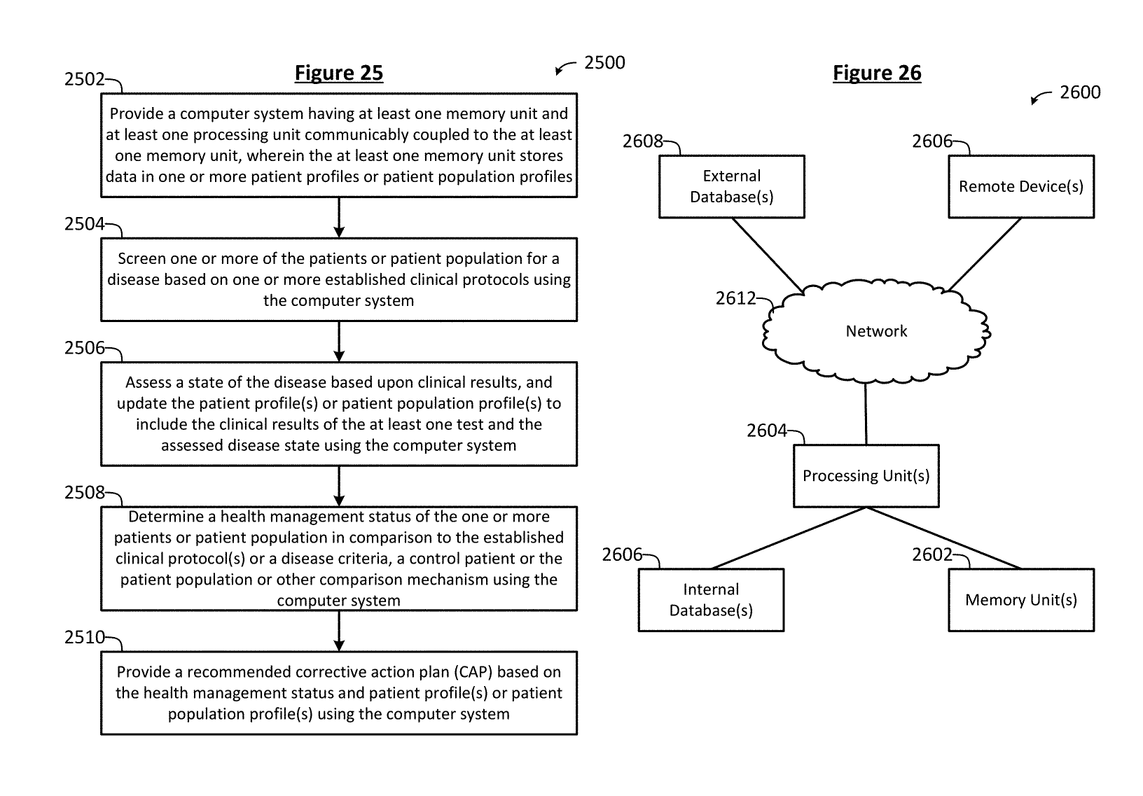

FIG. 25 is a flow chart of a method 2500 in accordance with another embodiment of the current invention. The computerized method 2500 of population health management in a captivated healthcare system (a) provides a computer system in block 2502 having at least one memory unit and at least one processing unit communicably coupled to the at least one memory unit, wherein the at least one memory unit stores data in one or more patient profiles or patient population profiles, (b) screens one or more of the patients or patient population for a disease based on one or more established clinical protocols using the computer system in block 2504, (c) assesses a state of the disease based upon clinical results, and updating the patient profile(s) or patient population profile(s) to include the clinical results of the at least one test and the assessed disease state using the computer system in block 2506, (d) determines a health management status of the one or more patients or patient population in comparison to the established clinical protocol(s) or a disease criteria, a control patient or the patient population or other comparison mechanism using the computer system in block 2508, and (e) provides a recommended corrective action plan (CAP) based on the health management status and patient profile(s) or patient population profile(s) using the computer system 2510. The captivated healthcare system can be a prison, nursing home, assisted living facility, veteran's facility, school, college, or university, etc.

In addition, the method may include the step of coordinating a delivery of a health care to a plurality of patients using the computer system, wherein the health care comprises an acute and non-acute integrated health care involving at least one of a physical health care, a behavioral health care, or a dental health care. Other steps may include (a) evaluating the delivery of the health care to the plurality of patients using the computer system based on one or more metrics comprising at least one of a patient access to the health care, a quality of the health care, a cost of the health care, a clinical data, a non-clinical data or a financial data, (b) coordinating a follow-up to the delivery of the health care to the plurality of patients using the computer system, or (c) providing one or more health education materials to the plurality of patients using the computer system. The patient profile(s) or patient population profile(s) may include at least one unique identifier such as medical record number, social security number, inmate number, unit number, facility name, region name, or state name. The patient profile(s) or patient population profile(s) may further includes a prior medical record(s) or a history of medical conditions.

The screening step may further (a) include performing at least one screening test and including results of the at least one screening test in the patient profile(s) or patient population profile(s), (b) be based on the established clinical protocol and/or disease criteria, or (c) include an evaluation of risk factors in the patient profile(s) or patient population profile(s) relevant to the disease state or condition. The disease may be a first disease, and the steps further include determining a health management status of the patient with respect to a second disease based on the patient profile and a co-morbidity of the first disease with the second disease.

Other steps may include (a) receiving an inquiry or request from a patient for related products, treatment, procedures, tests, education or counseling via the computer system, (b) providing a recommended action and a clinical or non-clinical follow-up via telehealth, (c) providing a recommended action to a recipient on an intranet or internet-based system, (d) displaying a recommended action on a visual enabled device comprising at least one of a computer, a smart phone, or tablet, (e) implementing the recommended corrective action plan (CAP) to address the disease state or condition, (f) providing the one or more metrics and one or more recommendations via one or more devices communicably coupled to the computer system, (g) displaying at least one of the metrics in a dashboard format on the one or more devices, or (h) providing a quality system and risk management program.

FIG. 26 is a block diagram of a system 2600 in accordance with another embodiment of the current invention. The system 2600 of population health management in a captivated healthcare system includes at least one memory unit 2602 to store a patient profile or a patient population profile including a patient data and a test results, and at least one processing unit 2604 communicably coupled with at least one memory unit 2602. The at least one processing 2604 unit (a) screens one or more of the patients or patient population for a disease based on one or more established clinical protocols, (b) assesses a state of the disease based upon clinical results, and updating the patient profile(s) or patient population profile(s) to include the clinical results of the at least one test and the assessed disease state, (c) determines a health management status of the one or more patients or patient population in comparison to the established clinical protocol(s) or a disease criteria, a control patient or the patient population or other comparison mechanism, and (d) provides a recommended corrective action plan (CAP) based on the health management status and patient profile(s) or patient population profile(s). The at least one processing unit 2604 can be communicably coupled to one or more internal databases 2606 or local devices. In addition, the at least one processing unit 2604 can be communicably coupled to one or more remote devices 2608 and/or external database(s) 2610 via a network 2612. Moreover, the at least one processing unit 2604 can automatically receive data, transmit data or control medical-related devices. The system 2600 may perform other functionality and/or include other components as described above in reference to FIG. 25.

To facilitate the understanding of this invention, a number of terms are defined below. Terms defined herein have meanings as commonly understood by a person of ordinary skill in the areas relevant to the present invention. Note that these terms may be used interchangeable without limiting the scope of the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, AB, BBC, AAABCCCC, CBBAAA, CABABB, and so forth. The skilled artisan will understand that typically there is no limit on the number of items or terms in any combination, unless otherwise apparent from the context.

It will be understood by those of skill in the art that information and signals may be represented using any of a variety of different technologies and techniques (e.g., data, instructions, commands, information, signals, bits, symbols, and chips may be represented by voltages, currents, electromagnetic waves, magnetic fields or particles, optical fields or particles, or any combination thereof). Likewise, the various illustrative logical blocks, modules, circuits, and algorithm steps described herein may be implemented as electronic hardware, computer software, or combinations of both, depending on the application and functionality. Moreover, the various logical blocks, modules, and circuits described herein may be implemented or performed with a general purpose processor (e.g., microprocessor, conventional processor, controller, microcontroller, state machine or combination of computing devices), a digital signal processor (“DSP”), an application specific integrated circuit (“ASIC”), a field programmable gate array (“FPGA”) or other programmable logic device, discrete gate or transistor logic, discrete hardware components, or any combination thereof designed to perform the functions described herein. Similarly, steps of a method or process described herein may be embodied directly in hardware, in a software module executed by a processor, or in a combination of the two. A software module may reside in RAM memory, flash memory, ROM memory, EPROM memory, EEPROM memory, registers, hard disk, a removable disk, a CD-ROM, or any other form of storage medium known in the art.

All of the systems, devices, computer programs, compositions and/or methods disclosed and claimed herein can be made and executed without undue experimentation in light of the present disclosure. While the systems, devices, computer programs, compositions and methods of this invention have been described in terms of preferred embodiments, it will be apparent to those of skill in the art that variations may be applied to the systems, devices, computer programs, compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

What is claimed is:

1. A computerized method of population health management in a captivated healthcare system, comprising:
providing a computer system having at least one memory unit and at least one processing unit communicably coupled to the at least one memory unit, wherein the at least one memory unit stores data in one or more patient profiles or patient population profiles;
screening one or more of the patients or patient population for a disease based on one or more established clinical protocols using the computer system;
assessing a state of the disease based upon clinical results, and updating the patient profile(s) or patient population profile(s) to include the clinical results of the at least one test and the assessed disease state using the computer system;
determining a health management status of the one or more patients or patient population in comparison to the established clinical protocol(s) or a disease criteria, a control patient or the patient population or other comparison mechanism using the computer system; and
providing a recommended corrective action plan (CAP) based on the health management status and patient profile(s) or patient population profile(s) using the computer system.

2. The method of claim 1, further comprising the step of coordinating a delivery of a health care to a plurality of patients using the computer system, wherein the health care comprises an acute and non-acute integrated health care involving at least one of a physical health care, a behavioral health care, or a dental health care.

3. The method of claim 2, further comprising the step of evaluating the delivery of the health care to the plurality of patients using the computer system based on one or more metrics comprising at least one of a patient access to the health care, a quality of the health care, a cost of the health care, a clinical data, a non-clinical data or a financial data.

4. The method of claim 2, further comprising the step of coordinating a follow-up to the delivery of the health care to the plurality of patients using the computer system.

5. The method of claim 1, further comprising the step of providing one or more health education materials to the plurality of patients using the computer system.

6. The method of claim 1, wherein the patient profile(s) or patient population profile(s) includes at least one unique identifier such as medical record number, social security number, inmate number, unit number, facility name, region name, or state name.

7. The method of claim 1, wherein the patient profile(s) or patient population profile(s) further includes a prior medical record(s) or a history of medical conditions.

8. The method of claim 1, wherein the screening step further comprises performing at least one screening test and including results of the at least one screening test in the patient profile(s) or patient population profile(s).

9. The method of claim 1, wherein the screening step is based on the established clinical protocol and/or disease criteria.

10. The method of claim 1, wherein the screening step further comprises an evaluation of risk factors in the patient profile(s) or patient population profile(s) relevant to the disease state or condition.

11. The method of claim 1, wherein the disease comprises a first disease, and further comprising the step of determining a health management status of the patient with respect to a second disease based on the patient profile and a co-morbidity of the first disease with the second disease.

12. The method of claim 1, further comprising the step of receiving an inquiry or request from a patient for related products, treatment, procedures, tests, education or counseling via the computer system.

13. The method of claim 1, further comprising the step of providing a recommended action and a clinical or non-clinical follow-up via telehealth.

14. The method of claim 1, further comprising the step of providing a recommended action to a recipient on an intranet or internet-based system.

15. The method of claim 1, further comprising the step of displaying a recommended action on a visual enabled device comprising at least one of a computer, a smart phone, or tablet.

16. The method of claim 1, further comprising implementing the recommended corrective action plan (CAP) to address the disease state or condition.

17. The method of claim 1, further comprising the step of providing the one or more metrics and one or more recommendations via one or more devices communicably coupled to the computer system.

18. The method of claim 17, further comprising the step of displaying at least one of the metrics in a dashboard format on the one or more devices.

19. The method of claim 1, further comprising the step of providing a quality system and risk management program.

20. The method of claim 1, wherein the captivated healthcare system comprises a prison, nursing home, assisted living facility, veteran's facility, school, college, or university.

21. A system of population health management in a captivated healthcare system, comprising:
at least one memory unit to store a patient profile or a patient population profile including a patient data and a test results;
at least one processing unit communicably coupled with at least one memory unit; and
the at least one processing unit (a) screens one or more of the patients or patient population for a disease based on one or more established clinical protocols, (b) assesses a state of the disease based upon clinical results, and updating the patient profile(s) or patient population profile(s) to include the clinical results of the at least one test and the assessed disease state, (c) determines a health management status of the one or more patients or patient population in comparison to the established clinical protocol(s) or a disease criteria, a control patient or the patient population or other comparison mechanism, and (d) provides a recommended corrective action plan (CAP) based on the health management status and patient profile(s) or patient population profile(s).

22. The system of claim 21, wherein the at least one processing unit further coordinates a delivery of a health care to a plurality of patients, wherein the health care comprises an acute and non-acute integrated health care involving at least one of a physical health care, a behavioral health care, or a dental health care.

23. The system of claim 22, wherein the at least one processing unit further evaluates the delivery of the health care to the plurality of patients based on one or more metrics comprising at least one of a patient access to the health care, a quality of the health care, a cost of the health care, a clinical data, a non-clinical data or a financial data.

24. The system of claim 22, wherein the at least one processing unit further coordinating a follow-up to the delivery of the health care to the plurality of patients.

25. The system of claim 21, wherein the at least one processing unit further provides one or more health education materials to the plurality of patients.

26. The system of claim 21, wherein the patient profile(s) or patient population profile(s) includes at least one unique identifier such as medical record number, social security number, inmate number, unit number, facility name, region name, or state name.

27. The system of claim 21, wherein the patient profile(s) or patient population profile(s) further includes a prior medical record(s) or a history of medical conditions.

28. The system of claim 21, wherein the screening provided by the at least one processing unit further comprises performing at least one screening test and including results of the at least one screening test in the patient profile(s) or patient population profile(s).

29. The system of claim 21, wherein the screening provided by the at least one processing unit is based on the established clinical protocol and/or disease criteria.

30. The system of claim 21, wherein the screening provided by the at least one processing unit further comprises an evaluation of risk factors in the patient profile(s) or patient population profile(s) relevant to the disease state or condition.

31. The system of claim 21, wherein the disease comprises a first disease, and further comprising the step of determining a health management status of the patient with respect to a second disease based on the patient profile and a co-morbidity of the first disease with the second disease.

32. The system of claim 21, wherein the at least one processing unit further receives an inquiry or request from a patient for related products, treatment, procedures, tests, education or counseling.

33. The system of claim 21, wherein the at least one processing unit further provides a recommended action and a clinical or non-clinical follow-up via telehealth.

34. The system of claim 21, wherein the at least one processing unit further provides a recommended action to a recipient on an intranet or internet-based system.

35. The system of claim 21, wherein the at least one processing unit further displays a recommended action on a visual enabled device comprising at least one of a computer, a smart phone, or tablet.

36. The system of claim 21, wherein the at least one processing unit further implements the recommended corrective action plan (CAP) to address the disease state or condition.

37. The system of claim 21, wherein the at least one processing unit further provides the one or more metrics and one or more recommendations via one or more devices communicably coupled to the system.

38. The system of claim 37, wherein at least one of the metrics is displayed in a dashboard format on the one or more devices.

39. The system of claim 21, wherein the at least one processing unit further providing a quality system and risk management program.

40. The system of claim 21, wherein the captivated healthcare system comprises a prison, nursing home, assisted living facility, veteran's facility, school, college, or university.

41. The system of claim 21, wherein the system is intranet or internet-based, displayed on a visual enabled device such as, but not limited to, a computer, smart phone, and tablet.

42. The system of claim 21, wherein the system utilizes a telehealth embedded function within the Electronic Medical Record to facilitate clinical and non-clinical communication.

43. The system of claim 21, wherein data for the patient profile(s) or patient population profile(s) is retrieved from a database externally linked to the system.

44. The system of claim 21, wherein data for the one or more metrics is retrieved from a database externally linked to the system.
